(19) United States
(12) Patent Application Publication
(10) Pub. No.: US 2025/0127901 A1
(43) Pub. Date: Apr. 24, 2025

(54) BIODEGRADABLE POLYMER-OIL BLENDS AND USES THEREOF

(71) Applicant: POLYGENE LTD., Jerusalem (IL)

(72) Inventors: Abraham Jacob Domb, Jerusalem (IL); Peter Siman, Eilaboun (IL)

(21) Appl. No.: 18/730,676

(22) PCT Filed: Jan. 30, 2023
(86) PCT No.: PCT/IL2023/050099

§ 371 Date: Jul. 19, 2024

Related U.S. Application Data
(60) Provisional application No. 63/267,344, filed on Jan. 31, 2022.

(51) Int. Cl. A61K 47/34; A61K 9/00; A61K 47/44; C08L 67/04

(52) U.S. Cl. CPC A61K 47/34; A61K 9/0024; A61K 47/44; C08L 67/04; C08L 2201/06; C08L 2203/02

(57) ABSTRACT

The invention generally contemplates homogeneous solid blends of at least one oil and at least one polymer, and uses thereof.

### TECHNOLOGICAL FIELD OF INVENTION

The invention generally concerns biodegradable materials for medical and non-medical applications.

### BACKGROUND OF THE INVENTION

Biodegradable polymers find applications as replacements and alternatives for petroleum-based plastics that are non-degradable and non-ecofriendly. The most common biodegradable polymers are polyesters of lactic acid, glycolic acid, caprolactone and combination thereof. Polymers such as polyesters, polylactide (PLA), poly(lactide-glycolide) (PLGA) and polycaprolactone (PCL) are commonly used in medicine and also find use in many other applications where degradability is needed.

To overcome drawbacks associated with the crystallinity of PLA, which makes PLA-based materials typically hard and brittle, PLA has been blended with various components to gain the required material properties. For example, PLA blends have been prepared with polymers, such as polycaprolactone, starch, poly(ethylene glycol) (PEG), and others. However, mixing the blends together is a tedious procedure due to lack of material compatibility. While addition of starch increases PLA's tensile strength and hydrophilicity, PLA flexibility is reduced due to the incompatibility between the two polymers. Considering the fact that high quality PLA plastic material is obtained without compromising its biodegradable properties, the interest of using vegetable oils as plasticizers have been in focus in recent years. It has been illustrated that vegetable oils could be chemically transformed (maleinized-, acrylated-, hydroxylated-, epoxidized-, etc) to prepare polymer-oil blends [1].

Epoxidized vegetable oils blended with a polymer changed the chemical structure of the polymer, resulting in a new material having high mechanical strengths and morphology useful in food packaging industries. Further, some of the epoxidized oils provide additional advantages as preservatives for polymers. For example, the use of pine oil offers antimicrobial activity on various microorganisms such as Micrococcus luteus and Bacillus subtilis, as well as antioxidant potential [2-10].

### REFERENCES

[1] María Jesús Garcíα-Campo, Teodomiro Boronat, Luis Quiles-Carrillo, Rafael Balart, Nestor Montanes, Manufacturing and Characterization of Toughened Poly(lactic acid) (PLA) Formulations by Ternary Blends with Biopolyesters, Polymers 10, 2018, 3.
[2] Y.Q. Xu, J. P. Qu, Mechanical and rheological properties of epoxidized soybean oil plasticized poly(lactic acid). J. Appl. Polym. Sci. 112, 2009, 3185-3191.
[3] Raina Jama Awale, Fathilah Binti Ali, Azlin Suhaida Azmi, Noor Illi Mohamad Puad, Hazleen Anuar, Azman Hassan, Enhanced Flexibility of Biodegradable Polylactic Acid/Starch Blends Using Epoxidized Palm Oil as Plasticizer, Polymers (Basel). 10, 2018, 977.
[4] Buong Woei Chieng, Nor Azowa Ibrahim, Yoon Yee Then, Yuet Ying Loo, Epoxidized Vegetable Oils Plasticized Poly(lactic acid) Biocomposites: Mechanical, Thermal and Morphology Properties, Molecules 19, 2014, 16024-16038.
[5] M. Llana-Ruiz-Cabello, S. Pichardo, J.M. Bermúdez, A. Baños, C. Núñez, E. Guillamón, S. Aucejo, A.M. Cameán, Development of PLA films containing oregano essential oil (Origanum vulgare L. virens) intended for use in food packaging, Food Addit Contam Part A Chem Anal Control Expo Risk Assess. 33, 2016, 1374-1386.
[6] Mónica Hernández-López, Zormy Nacary Correa-Pacheco, Silvia Bautista-Baños, Leonor Zavaleta-Avejar, José Jesús Benítez-Jiménez, Marcos Antonio Sabino-Gutiérrez, Pedro Ortega-Gudiño, Materials Chemistry and Physics, 2019.
[7] Jose M. Ferri, Daniel Garcia-Garcia, Nestor Montanes, Octavio Fenollar, Rafael Balart, The effect of maleinized linseed oil as biobased plasticizer in poly(lactic acid)-based formulations, PolymInt 66, 2017, 882-891.
[8] Alfredo Carbonell-Verdu, M. Dolores Samper, Daniel Garcia-Garcia, Lourdes Sanchez-Nacher, Rafael Balart, Plasticization effect of epoxidized cottonseed oil (ECSO) on poly(lactic acid), Industrial Crops and Products, 104, 2017, 278-286.
[9] Buong Woei Chieng, Nor Azowa Ibrahim, Yoon Yee Then, Yuet Ying Loo, Mechanical, Thermal, and Morphology Properties of Poly(lactic acid) Plasticized With Poly(ethylene glycol) and Epoxidized Palm Oil Hybrid Plasticizer, Polymer Engineering and Science, 56, 2016, 1169-1174.
[10] V. S. Giita Silverajah, Nor Azowa Ibrahim, Wan Md Zin Wan Yunus, Hazimah Abu Hassan, Chieng Buong Woei, A Comparative Study on the Mechanical, Thermal and Morphological Characterization of Poly(lactic acid)/Epoxidized Palm Oil Blend, Int. J. Mol. Sci. 13, 2012, 5878-5898.

### SUMMARY OF THE INVENTION

Fiducial marker placement is often used to improve upon the accuracy of cutting edge, state-of-the-art techniques, such as minimally invasive surgery, interventional procedures and precision brachytherapy. Clinical scenarios include the need for marking of abnormal imaging findings or tumors, most commonly in the breast, to permit identification at surgery and the need for multiple three-dimensional markers for precise serial application of stereotactic radiation. Currently, these markers are most often metallic making them permanent. Yet, permanency of the markers is often not required. For example, if management is surgical, markers are required for in situ identification for at most a range of few weeks, and if radiation treatment is considered, the marker is usually required for only a 2-3-month period, and most often not exceeding a 6-month course of therapy. Nevertheless, retention of a foreign body beyond this time frame is extraneous and sometimes potentially undesirable. Particularly, it is crucial to minimize obscuring image artefacts, particularly in the region most likely to demonstrate recurrence and metallic implants are known to exhibit a strong blooming artefact on magnetic resonance (MR) imaging.

The inventors of the technology disclosed herein have now developed a novel blend material and demonstrated its superiority as a fiducial marker, that is not only biocompatible and biodegradable, but which can also be easily manufactured as a drug or material depot in any shape and size. The blend material or product of the invention is a solid single-phase polymer material formed of a blend of oils and polymers, which exhibits properties that, despite presence of a high oil content, are similar to or substantially identical to properties of the original polymer. In other words, against the expected, properties of the polymer have been maintained in the blend as a whole, despite the high oil content and the homogenous distribution of the oil in the polymer. More so, the blend is a storage-stable material, demonstrating material continuity and homogeneity with no separation or change in the blend properties, nor in its integrity, composition and structure, even after many months of storage at room temperature. The long periods of mechanical, physical and chemical stability render blends of the invention suitable as medical implants and as in vivo fiducial markers or tags.

Blends of this invention are suitable for medical and non-medical uses. They can be used as biodegradable plastics, or bioplastics, for any application that current non-degradable plastics such as polyethylene and polypropylene are used. They can be used as disposable syringes and medical supplies, boards, coatings, rods, 3D printing inks, containers, trays, sheets, foams, packaging materials or any other object or product. The bioplastics of this invention have applications in the paper industry, agriculture, food, automotive, aviation, aerospace, electronic devices, medical devices and more. The addition of low-cost oil to the biodegradable aliphatic polymers such as PLA, PLGA, PCL, and polycarbonate, reduces their cost, rendering them highly economic for bioplastic applications.

Typically, solid blends of the invention may contain between about 10 and 60 wt % oil and between 40 and 90 wt % of the polymer, and may further contain one or more additives or actives of various types. The maximal amount of the oil to be incorporated in the polymer, may be varied to produce a gamut of products of various properties and capabilities. Blends of the invention have been thus formed into a variety of different blend compositions, and further into a variety of different solid forms or objects, including uniform films, molded elements, microspheres, nanospheres, coatings, medical tools, tags or clips and other objects of different shapes and sizes, as disclosed herein.

Thus, in most general terms, the invention provides a solid single-phase blend of at least one polymer and at least one oil, the single-phase blend having one or more of the following properties:
high oil content;
homogeneous distribution of oil within the polymer;
does not exhibit oily behavior;
does not leak oil over time;
maintains structural and compositional integrity over time;
maintains storage and in vivo stabilities;
maintains properties of the polymer;
moldable solid material;
non-toxic;
provides a polymeric composition with certain properties of the incorporated oil such as visibility under imaging devices when incorporating oily contrast agent such as Lipiodol or become antimicrobial when essential oils are used;
allow incorporation of oil soluble drugs and agents uniformly to the polymer composite; and
can be manufactured in various shapes, nano and microparticles, fibers, strip, films and slabs.

In a first of its aspects, the invention provides a homogeneous solid blend of at least one oil and at least one polymer, wherein the blend having at least one mechanical and/or physical and/or chemical property substantially identical to the mechanical and/or physical and/or chemical property of the at least one polymer alone.

Further provided is a solid blend of at least one oil and at least one polymer, wherein the blend is a single-phase solid material, i.e., at any temperature.

The invention further provides a solid single-phase blend of at least one oil and at least one polymer, the blend comprising between 10 and 60 wt % of the at least one oil and optionally one or more active or non-active additives.

A blend or a product of the invention may also be characterized as a polymeric matrix physically holding or encompassing or comprising at least one oil, wherein the polymeric matrix and the at least one oil form a single-phase solid material having at least one mechanical or physical or chemical characteristic that is identical to a characteristic of a polymeric material making up (being or comprised in) the polymeric matrix.

As disclosed herein, products of the invention, being solid single-phase materials are composed of a polymer and an oil that in combination form a solid single-phase product, without undergoing any chemical bonding. The interaction between the polymer and the oil is substantially physical. Despite the high oil content and the single-phase characteristic of the solid product, one or more of the properties of the polymer are not lost. The blend material exhibits at least one mechanical and/or physical property that is “substantially identical to the mechanical and/or physical property of the at least one polymer alone”. In other words, at least one of the following mechanical and physical characteristics of the at least one polymer and the blend comprising same are substantially the same: modulus of elasticity, tensile strength, elongation, hardness, fatigue limit, melting temperature, visibility and feeling, and film formation ability. For example, where a mechanical or physical property of the polymer alone (not in combination with the oil) has a value X or a characteristic X, the same property of the blend (polymer and oil in the single-phase) has a value or a characteristic that is also X or substantially X.

A difference in the mechanical or physical property of the blend or the polymer or an identity between the properties is determined by carrying out a measurement of the property of each of the blend and polymer under identical conditions or under an identical test protocol.

In some embodiments, the mechanical or physical property of the blend product that is substantially identical to the same mechanical or physical property of the at least one polymer alone is melting temperature. In other words, the melting temperature of the blend and that of the polymer making up the blend is substantially identical, or substantially same.

Each of the mechanical or physical properties may be determined by utilizing acceptable measuring protocols and apparatuses. For determining identity between the mechanical or physical properties, same protocols and apparatuses should be used. For example, the materials' melting temperatures may be determined by a Thiele tube, an electronic melting point apparatus, Fisher-Johns apparatus, Gallenkamp (Electronic) apparatus, differential scanning calorimetry (DSC) and others, as known in the art.

The term “substantially identical” means that a difference between two values relating to the same mechanical or physical property, one value characteristic of the at least one polymer alone and the other value characteristic of the blend comprising the same at least one polymer, does not exceed 20%, 15%, 10%, 9%, 8%, 7%, 6%, 5%, 4%, 3%, 2%, 1%, 0.5%, 0.1%, or 0.05%. In some embodiments, the term ‘substantially identical’ means ‘identical’.

In some embodiments, the invention provides a homogeneous solid blend of at least one oil and at least one polymer, wherein the blend having a melting temperature that is substantially the same as the melting temperature of the at least one polymer when measured alone. For purposes herein, the melting temperature may be determined by any method known in the art or any of the methods disclosed herein.

In some embodiments, the melting temperature of both the blend and the polymer itself is determined by differential scanning calorimetry (DSC). In some embodiments, the melting temperature is determined as each of the blend and polymer were heated under increased temperature from 25 to 200° C. at a rate of 10° C./min. In some embodiments, the melting temperature is measured on a DSC-1, Mettler Toledo instrument, under a nitrogen atmosphere, e.g., wherein each sample (blend alone and polymer alone) was contained in a separate aluminium crucible and heated from 25 to 200° C. at a scan rate of 10° C./min.

The invention further provides a blend of at least one oil and at least one polymer, the blend being formed by applying a shear force onto a mixture comprising an amount of the at least one polymer and an amount of the at least one oil, optionally in the presence of at least one solvent. Processes for manufacturing and for processing a blend of the invention are provided hereinbelow.

As disclosed herein, products of the invention are solid single-phase blends of a polymer and an oil. The term “solid single-phase blend” suggests a homogeneous solid mixture of the polymer and oil, which at room temperature (25-30° C.) and atmospheric pressure appears as one continuous phase without any visible particles or different material regions. Any part or region of the solid blend substantially comprises the same composition, exhibiting identical properties as any other part or region of the solid blend. The single-phase properties may also be determined spectroscopically or by high-resolution techniques.

The blend is a physical mixture of the oil and polymer, wherein the two materials do not exhibit any chemical bonding therebetween or with any other material that may be present, e.g., an active or a non-active additive.

The at least one polymer is typically a biodegradable polymer, which may be a solid or a semi-solid or a liquid, capable of undergoing deterioration and complete degradation when exposed to certain conditions, which may be uniquely important for medical, cosmetic, agricultural, environmental and related applications. The at least one polymer may be a natural biodegradable polymer or a synthetic or a semisynthetic biodegradable polymer. Of particular interest are aliphatic polyesters that may include polyglycolide (PGA), polylactide (PLA), polycaprolactone (PCL), and their copolymers, including poly(lactic-glycolic acid) (PLGA), and poly(lactic acid-caprolactone) copolymers (PLCL).

In some embodiments, a blend of the invention is a homogeneous solid blend of at least one oil and at least one aliphatic polyester, wherein the blend having one or more mechanical and/or physical properties, e.g., melting temperature, substantially identical to the same mechanical and/or physical properties, e.g., melting temperature, of the at least one aliphatic polyester alone.

In some embodiments, the aliphatic polyester is selected from polyglycolide (PGA), polylactide (PLA), polycaprolactone (PCL), polylactic-glycolic acid copolymer (PLGA) and other copolymers of PGA, PLA (including d-PLA, 1-PLA, DI-PLA and meso-PLA) or PCL.

The at least one polymer, e.g., aliphatic polyester, is selected based on its molecular weight. Typically, polymers used in accordance with the invention have chain length of 10-10,000 monomer units. In some embodiments, the polymers have between 10 and 9,000, 10 and 8,000, 10 and 7,000, 10 and 6,000, 10 and 5,000, 10 and 4,000, 10 and 3,000, 10 and 2,000 10 and 1,000, 100 and 10,000, 100 and 9,000, 100 and 8,000, 100 and 7,000, 100 and 6,000, 100 and 5,000, 100 and 4,000, 100 and 3,000, 100 and 2,000, 100 and 1,000, 1,000 and 10,000, 1,000 and 9,000, 1,000 and 8,000, 1,000 and 7,000, 1,000 and 6,000, 1,000 and 5,000, 1,000 and 4,000, or between 1,000 and 3,000. In some embodiments, the polymer used in blends of the invention comprises between 100 and 3,000, 100 and 2,000 or 100 and 1,000 connector units or monomer units.

In some embodiments, the at least one polymer is an aliphatic polyester having a chain length of between 10 and 10,000 monomer units, as optionally selected as above.

In some embodiments, a blend of the invention is a homogeneous solid blend of at least one oil and at least one aliphatic polyester having between 10 and 10,000 repeating units, wherein the blend having a melting temperature, substantially identical to the melting temperature of the at least one aliphatic polyester alone.

The at least one oil may be any fatty material which may be naturally derived, a synthetic or a semisynthetic oil material. The oil may be a naturally derived oil selected from triglycerides, fatty acids, fatty alcohols, fatty esters, vegetable oils and naturally occurring waxes and paraffins. Synthetic or semisynthetic oils may be similarly selected from mono-, di- and triglycerides, fatty acids, fatty alcohols, fatty esters, paraffins, iodinated oils, modified oils, fully synthetic oils, hydrogenated vegetable oils and waxes. The vegetable oil may be selected from avocado oil, Brazil nut oil, Canola oil, coconut oil, corn oil, cottonseed oil, linseed oil, grape seed oil, hemp seed oil, olive oil, palm oil, peanut oil, poppyseed oil, sesame oil, soybean oil, walnut oil, sunflower oil, cardanol oil and others.

In some embodiments, the oil is a vegetable oil selected from castor oil, olive oil, soybean oil, sesame oil, palm oil, cardanol oil and poppyseed oil.

In some embodiments, the oil is an iodinated oil. The iodinated oil may be any oil which has been treated with iodine or hydriodic acid to form the iodized equivalent. The iodinated oil is typically an iodinated vegetable oil, such as iodinated poppyseed oil.

In some embodiments, the oil is an ethiodized form of an ethyl ester of a fatty acid, e.g., an ethyl ester of a fatty acid of poppyseed oil. In some embodiments, the oil is an ethyl monoiodostearate or ethyl diiodostearate.

In some embodiments, the oil is Lipiodol (iodized poppyseed oil), i.e., a medical contrast agent.

In some embodiments, the oil is a fatty acid or a fatty alcohol or a fatty ester having a fatty saturated or unsaturated acid or alcohol carbon chain (or both), wherein the saturated or unsaturated acid or alcohol carbon chain having between 8 and 18 carbon atoms.

Non-limiting examples of fatty acids include caprylic acid, capric acid, lauric acid, myristic acid, palmitic acid, stearic acid, myristoleic acid, palmitoleic acid, sapienic acid, oleic acid, elaidic acid, vaccenic acid, linoleic acid, linoelaidic acid, α-linolenic acid, and others.

Non-limiting examples of fatty alcohols include capryl alcohol, pelargonic alcohol, capric alcohol, hendecanol, lauryl alcohol, isotridecanol, myristyl alcohol, pentadecyl alcohol, cetyl alcohol, palmitoleyl alcohol, heptadecanol, stearyl alcohol, oleyl alcohol and others.

Non-limiting examples of fatty esters include monoglycerides, diglycerides, or triglycerides of any of the fatty acids and/or fatty alcohols disclosed herein.

In some embodiments, the at least one oil is medium chain triglycerides (MCT). The MCT may be formed of medium chain fatty acids or fatty alcohols having aliphatic chains of 6 to 12 carbon atoms.

In some embodiments, the oil is a hydrogenated fatty acid, fatty alcohol or fatty ester.

In some embodiments, the oil is a hydrogenated vegetable oil derived from any vegetable oil known in the art.

In some embodiments, the hydrogenated vegetable oil is a partially or a fully hydrogenated di- or triglyceride.

Waxes may also be used as at least one oil. The wax may be a plant wax, an animal wax, or a modified wax material. The waxes may be selected from carnauba wax, vegetable wax, beeswax, coconut wax, Candelilla wax, soy wax and others.

In some embodiments, the at least one wax is carnauba wax or vegetable wax or beeswax or coconut wax or Candelilla wax or any other wax known in the art.

The wax may be a modified wax, namely a conjugate of at least one wax and a material selected from hydrocarbons, polysaccharides, proteins, amino acids, aliphatic materials, lipids, and various polymers.

The amount of the oil in the solid blend of the invention is at least 10 wt %. The amount typically does not exceed 60 wt %. In some embodiments, the amount of the at least one oil is between 10 and 60 wt %, between 10 and 50 wt %, between 10 and 40 wt %, between 10 and 30 wt %, between 10 and 20 wt %, between 20 and 50 wt %, between 20 and 40 wt %, between 20 and 30 wt %, between 30 and 50 wt %, between 30 and 40 wt %, or between 40 and 50 wt %.

In some embodiments, a blend of the invention is a homogeneous solid blend of at least one oil and at least one aliphatic polyester, wherein the blend having a mechanical and/or physical property, e.g., melting temperature, substantially identical to the same mechanical and/or physical property, e.g., melting temperature, of the at least one aliphatic polyester alone, and wherein the at least one oil is selected from naturally derived oil such as triglycerides, fatty acids, fatty alcohols, fatty esters, vegetable oils and naturally occurring waxes; or synthetic or semisynthetic oils which may be selected from triglycerides, fatty acids, fatty alcohols, fatty esters, paraffins, iodinated oils, modified oils, fully synthetic oils, hydrogenated vegetable oils and waxes.

The at least one oil is selected based on the purpose and intended use of the solid blend. In some embodiments, the oil may be selected amongst liquids and solids having a melting temperature below 50° C. In some embodiments, the at least one oil is selected amongst naturally derived oils, such as triglycerides, fatty acids, fatty alcohols, fatty esters, vegetable oils and naturally occurring waxes; or synthetic or semisynthetic oils which may be selected from triglycerides, fatty acids, fatty alcohols, fatty esters, paraffins, iodinated oils, modified oils, fully synthetic oils, hydrogenated vegetable oils and waxes, provided that the at least one oil having a melting temperature below 50° C. In some embodiments, the oil is a saturated oil, such as hydrogenated vegetable oil or medium chain triglycerides, such as Migliol.

Depending on a variety of factors, including, inter alia, the oil to be incorporated, the polymer structure and its molecular weight, and others, the ratio polymer-to-oil may be varied. The mass ratio between the polymer and the oil (polymer: oil) may be between 10:1 to 1:10, or between 9:1 to 4:6. In some embodiments, the amount of the oil in the blend exceeds the amount of the polymer. In other embodiments, the amount of the polymer in the blend exceeds the amount of the oil.

In some embodiments, the oil content in the polymer blend may be between 10 and 60% or between 10 and 40% w/w, or selected herein. In some embodiments, the oil content in the blend is between 10 and 40% w/w.

Blends of the invention are novel biomaterials which may be used for manufacturing a variety of objects, devices or tools usable in therapeutic, cosmetic, agricultural applications or in any other technological field requiring material delivery or release over time. The versatility of products of the invention from a point of view of both stability and biocompatibility, and the ability to incorporate into the blends additional active or non-active agents, makes products of the invention particularly useful in the medical arena, e.g., as diagnostic markers, or as biocompatible and biodegradable implants, or as biocompatible and biodegradable drug depots, e.g., for instillation in vivo. Products of the invention may be shaped into any desirable shape and size, for example into a form of nanoparticles, microparticles, films, fibers, slabs, sheets, rods, plates, filaments, shape memory compositions, sachets, capsules and other shaped objects, and may be made to comprise a variety of or additives. The additives may be selected amongst additives useful in treatment, prevention or diagnosis of a human or an animal body or amongst such additives that modify, typically improve, at least one mechanical or physical attribute of the product, or additives which are suitable for cosmetic or agricultural uses.

In some embodiments, the additive is a non-active agent tailored to endow a product of the invention with one or more property such as relating to the product mechanical properties, appearance, stability, degradability, final use or intended use, etc.

In some embodiments, the additive may be a drug, an active agent that is suitable for use in agriculture such as an herbicide, insecticide and fungicide, a contrast agent, a metal oxide, a colorant, a binder, a solubilizing agent, a disintegrant, a lyophilizing agent, a stabilizer, an adhesive material, a surfactant, a toxicity reducing agent, a heat conducting agent, a radiation responsive element, a magnetic material (or particles), and others.

In some embodiments, the additive may be an active such as a pharmaceutically acceptable agent, a biomolecule, a small molecule, biomolecules, oligonucleotides, genes, carbohydrates, angiogenic factors, cell cycle inhibitors, hormones, nucleotides, amino acids, sugars, lipids, stem cells, antibiotic agents, antimicrobial agents, antifungal agents, anti-inflammatory agents, antioxidative agents, anti-proliferative agents, anticancer agents, and others.

In some embodiments, the additive is mixed within the polymer-oil blend or provided encapsulated in nanoparticles, nanocapsules, microparticles, microcapsules, liposomes, micelles, vesicles, or any other known material delivery vehicle. In other words, a blend may comprise a plurality of vehicles, such as those mentioned above, which contain one or more additives. As such, the nature of the additive may vary. Both hydrophilic and hydrophobic or lipophilic materials may be embedded or contained within the blend. For example, nanocapsules containing one or more hydrophilic/hydrophobic materials may be provided in the blend, while hydrophobic/hydrophilic materials may be contained in the oil making up the blend. Such a distribution of materials of different characteristics permits tailoring of various platforms of material release, i.e., controlled release, sustained release, immediate release etc.

Products of the invention may be used as implantable material, e.g., drug, depots for enabling release, e.g., controlled release, sustained release or immediate release, of a material, e.g., a drug, or an oil contained in the blend. Release of the material, e.g., drug, may follow any one or more release profiles, which may depend, inter alia, on the type of drug or agent used, its compatibility with the oil component, whether the drug is encapsulated (or in a vehicle as disclosed herein) or provided in an unencapsulated form, and others. The material, e.g., drug, may be a hydrophilic material or drug, a hydrophobic material or drug or an amphiphilic material or drug. Alternatively, the material or drug may be a combination of two or more such materials selected amongst hydrophilic materials or drugs, amongst hydrophobic materials or drugs or which may be a mixture of materials or drugs (wherein, for example, one material or drug is a hydrophilic material or drug and another is a hydrophobic material or drug). Thus, blends of the invention may comprise:
a hydrophilic material or drug in an encapsulated or generally capsule carrier;
a hydrophilic material or drug in an unencapsulated form;
a hydrophobic material or drug;
a hydrophobic material or drug encapsulated;
a mixture of any of the above.

Products of the invention may be implemented as implants of any form, size and shape and may be provided with a variety of therapeutic or diagnostic additives. The implants may be any medical or cosmetic implant that is provided to replace or support or enhance an existing biological or physiological structure, such as a tissue. The implants are typically intended to be inserted under a subject's skin or within a body natural cavity or a cavity formed during a surgical procedure, e.g., a biopsy. One such implant is a fiducial marker such as a diagnostic marker or a biopsy marker or a clip.

Fiducial markers are objects structured and formed for placing or instilling in a subject's body in the field of view of an optical tracking system for use as a point of reference or as a marking element preceding or following a medical procedure. The fiducial marker may be a biopsy marker or clip.

Biopsy markers are small elements that are used to identify a location of a soft tissue. Typically, markers are placed in or in a vicinity of a soft tissue immediately following a biopsy procedure. The marker is shaped and sized so that it can be easily identified at any time after the procedure using any imaging equipment. Solid markers of the invention, formed of blends of the invention, are not only detectable using a variety of imaging techniques, due to the presence of a contrasting agent, but are also biocompatible and biodegradable. Biopsy clips or markers of the invention may thus be shaped and sized to adopt a predetermined structures and may further comprise agents or additives as disclosed herein, such as iodinated oils, e.g., iodinated poppyseed oil or lipiodol, that can be detected by conventional imaging techniques, such as CT, MRI, mammography or other radiological techniques.

The invention thus provides an implant (for therapeutic, diagnostic or cosmetic use) formed of or comprising a homogeneous solid blend of at least one oil and at least one aliphatic polyester, wherein the blend having at least one mechanical and/or physical property substantially identical to the same mechanical and/or physical property of the at least one aliphatic polyester alone, and wherein the at least one oil is selected from naturally derived oils, such as triglycerides, fatty acids, fatty alcohols, fatty esters, vegetable oils and naturally occurring waxes; or synthetic or semisynthetic oils which may be selected from triglycerides, fatty acids, fatty alcohols, fatty esters, paraffins, iodinated oils, modified oils, fully synthetic oils, hydrogenated vegetable oils and waxes.

In some embodiments, the implant is a biopsy clip or a biopsy marker.

The clip or biopsy marker may be implanted or instilled in a subject's body by a needle or an instillation device. The clip may be used for marking a tissue site, typically a soft tissue, e.g., for the purpose of localization of a site that needs to be inspected or operated on. The tissue may be a breast tissue or a tissue of the gastrointestinal (GI) tract, which is marked during biopsy and imaged by, e.g., mammography, CT, MRI or ultrasound.

In some embodiments, the clip or marker may be placed in or in a vicinity of the soft tissue immediately following a biopsy procedure.

In some embodiments, the clip or marker is implanted under imaging and/or identified post-instillation by imaging.

In some embodiments, the clip or marker is coated with a hydrogel or a mucoadhesive material, to, e.g., increase biocompatibility with the soft tissue. The hydrogel may be or may comprise hyaluronic acid, collagen, polyethylene glycol or other hydrogel materials which may or may not be crosslinked.

In some embodiments, the clip or marker is implanted for a period of 1 or more month, 3 or more months, or 6 or more months, or 12 or more months. The period of residence in the body may be tailored such that the clip or marker biodegrades and eliminates from the site of instillation and the body at the end of said time period.

The clip or marker may comprise one of more active agents, such as drugs, and/or one or more contrast agents. The contrast agent may be any such material known and used in imaging a tissue in a subject's body. In some embodiments, the contrast agent is an iodinated oil. The iodinated oil may be any oil which has been treated with iodine or hydriodic acid to form the iodized equivalent. The iodinated oil is typically an iodinated vegetable oil, such as iodinated poppyseed oil.

In some embodiments, the contrast agent is an ethiodized form of an ethyl ester of a fatty acid, e.g., an ethyl ester of a fatty acid of poppyseed oil. In some embodiments, the contrast agent is an ethyl monoiodostearate or ethyl diiodostearate.

In some embodiments, the contrast agent is Lipiodol (iodized poppyseed oil).

In some embodiments, the contrast agent is diatrizoate, metrizoate, iothalamate, ioxaglate, iopamidol, iohexol, ioxilan, iopromide, iodixanol, iobitridol, ioversol, gadoterate, gadobutrol, gadoteridol, gadopentetate, gadobenate, gadopentetic acid dimeglumine, gadoxentate, gadoversetamide, gadodiamide, and others.

In some embodiments, the blend comprises at least one aliphatic polyester and lipiodol or iodinated poppy seed oil.

In some embodiments, the invention provides an implant in the form of a rod or film or a microsphere or a nanoparticle (for therapeutic, diagnostic, theragnostic or cosmetic use) formed of or comprising a homogeneous solid blend of at least one oil and at least one polymer, wherein the blend having mechanical and/or physical properties substantially identical to the same mechanical and/or physical properties of the at least one polymer alone;

wherein the at least one oil is selected from naturally derived oil such as triglycerides, fatty acids, fatty alcohols, fatty esters, vegetable oils and naturally occurring waxes; or synthetic or semisynthetic oils which may be selected from triglycerides, fatty acids, fatty alcohols, fatty esters, paraffins, iodinated oils, modified oils, fully synthetic oils, hydrogenated vegetable oils and waxes; and wherein the at least one polymer is a homopolymer or a copolymer of lactic acid, glycolic acid, caprolactone and di- and tri-methylene carbonate.

In some embodiments, the polymer is one or more of the commercially available polymers such as copolymers of lactic acid with glycolic acid, PLGA, at a molar ratio of 50:50, 75:25, 85:15; copolymers of lactic acid with caprolactone at 75:25 ratio; or homopolymers of polydioxanone, poly(trimethylene carbonate) and polyhydroxy butyrate) and their copolymers with hydroxy acids.

In some embodiments, Lipiodol and iron oxide are added in an amount of 30 and 0.2% w/w per PLGA75: 25 solution.

Blends of the invention comprising at least one polymer and at least one oil, and optionally other materials, are formed into a single-phase or a homogeneous solid mixture under conditions which, despite the high oil content, maintain one or more of the physical or mechanical properties of the polymer. By application of shear forces, as disclosed herein, which do not cause or bring about chemical association between the oil and polymer components, nor impose any structural or chemical change to either material, at least one of the mechanical or physical properties of the polymer remains substantially the same, even in the presence of the oil, and more so, even when the oil constitutes a substantive part of the blend as a whole. In other words, while the blend material is of a different composition from the polymer itself, the blend mechanical or physical properties may remain substantially identical to the properties of the polymer.

Thus, the invention further provides a process for preparing a blend material according to the invention, the process comprising treating a blend of at least one oil and at least one polymer, under a shear force, optionally in the presence of a solvent, at a temperature between room temperature (25-30° C.), and the melting temperature of the at least one polymer.

The melting temperature of the at least one polymer may vary based on the elected polymer. Generally speaking, the polymer melting temperature is between 50 and 200° C. Thus, in some embodiments, the process comprises treating a blend of at least one oil and at least one polymer, optionally in the presence of a solvent, at a temperature between room temperature and 200° C.

The shear forces applied may be achievable of blending, mixing, homogenization, or vortexing, utilizing any blender unit, mixer, homogenizer, rotor-stator, vortex device and others. In some embodiments, shear forces are applied at 10,000-50,000 rpm. In some embodiments, shear forces are applied at 15,000-50,000 rpm, 15,000-45000 rpm, 15,000-40,000 rpm, 15,000-35,000 rpm, 15,000-30,000 rpm, 20,000-50,000 rpm, 20,000-45,000 rpm, 20,000-40,000 rpm or between 19,000-26,000 rpm.

The shear forces do not cause or bring about chemical association between the oil and polymer components, nor impose any structural or chemical change to either material. In some embodiments, the shear force or the period of application of said force may be selected to achieve at least one mechanical or physical property that is substantially the same as the same mechanical or physical property measured or demonstrated for the polymer alone.

In some embodiments, the process comprises treating a blend of at least one oil and at least one polymer, in the absence of a solvent, at a temperature between room temperature and the melting temperature of the at least one polymer, wherein the treating comprises application of shear forces to the blend until a homogenous single-phase blend is obtained and cooling the blend to obtain the solid blend of the invention.

In some embodiments, the temperature may be a few degrees, e.g., 5-20° C., above the melting temperature of the polymer so it is melted to allow mixing with the oil and other components. The melt blend is cooled to room temperature to form the solid blend.

In some embodiments, the process comprises treating a blend of at least one oil and at least one polymer, in the presence of a solvent, at a temperature between room temperature and the melting temperature of the at least one polymer, wherein the treating comprises application of shear forces to the blend until complete or substantially complete evaporation of the solvent and formation of a homogenous single-phase blend, and cooling the blend to obtain the solid blend of the invention.

In some embodiments, treating comprises homogenizing the mixture. In some embodiments, homogenization may be achieved at 10,000-50,000 rpm. In some embodiments, homogenization may be achieved at 15,000-50,000 rpm, 15,000-45000 rpm, 15,000-40,000 rpm, 15,000-35,000 rpm, 15,000-30,000 rpm, 20,000-50,000 rpm, 20,000-45,000 rpm, 20,000-40,000 rpm or between 19,000-26,000 rpm.

In some embodiments, treating comprises homogenizing the mixture followed by continuous stirring (e.g., at between 500 and 1,500 rpm) for a time period sufficient to cause solvent evaporation.

In processes of the invention where a solvent is used, the solvent may be a volatile organic solvent such as chloroform, methylene chloride and ethyl acetate.

In some embodiments, a process of the invention comprises forming a mixture of at least one polymer, at least one oil and optionally at least one solvent and further optionally at least one additive.

In some embodiments, additives such as active agents are incorporated into the blend mixture, either in blend melt or solution or being incorporated after the blend formation.

The formed polymer-oil blend may be further treated or manipulated, e.g., by molding, injection molding, casting or by any other means, to transform the blend into filaments or sheets or rods or microparticles or nanoparticles or into any object of any shape and size.

Products of the invention generally encompass blends, devices, tools, implants, particles of any sort, and generally any object formed of a homogeneous solid blend of at least one oil and at least one polymer, wherein the blend has at least one mechanical and/or physical property substantially identical to the mechanical and/or physical properties of the at least one polymer alone.

Generally, the invention provides a homogeneous solid blend of at least one oil and at least one polymer, the blend having at least one mechanical and/or physical property substantially identical to the mechanical and/or physical properties of the at least one polymer alone.

In some configurations of blends of the invention, the blend comprising between 10 and 60 wt % of the at least one oil and optionally one or more active or non-active additives.

In some configurations of blends of the invention, the mechanical or physical property of the blend product that is substantially identical to the mechanical or physical property of the at least one polymer alone is melting temperature.

In some configurations of blends of the invention, the blend is a homogeneous solid blend of at least one oil and at least one polymer, wherein the blend having a melting temperature that is substantially the same as the melting temperature of the at least one polymer when measured alone.

In some configurations of blends of the invention, the at least one polymer is a biodegradable polymer.

In some configurations of blends of the invention, the at least one polymer is a natural biodegradable polymer or a synthetic or a semisynthetic biodegradable polymer.

In some configurations of blends of the invention, the at least one polymer is an aliphatic polyester.

In some configurations of blends of the invention, the aliphatic polyester is selected from polyglycolide (PGA), polylactide (PLA), polycaprolactone (PCL), and their copolymers.

In some configurations of blends of the invention, the aliphatic polyester is poly(lactic-glycolic acid) (PLGA) or poly(lactic acid-caprolactone) copolymers (PLCL).

In some configurations of blends of the invention, the aliphatic polyester has a chain length of 10-10,000 monomer units.

In some configurations of blends of the invention, the number of monomer units is between 10 and 9,000, 10 and 8,000, 10 and 7,000, 10 and 6,000, 10 and 5,000, 10 and 4,000, 10 and 3,000, 10 and 2,000 10 and 1,000, 100 and 10,000, 100 and 9,000, 100 and 8,000, 108 and 7,000, 100 and 6,000, 100 and 5,000, 100 and 4,000, 100 and 3,000, 100 and 2,000, 100 and 1,000, 1,000 and 10,000, 1,000 and 9,000, 1,000 and 8,000, 1,000 and 7,000, 1,000 and 6,000, 1,000 and 5,000, 1,000 and 4,000, or between 1,000 and 3,000.

In some configurations of blends of the invention, the at least one oil is a naturally derived, a synthetic or a semisynthetic oil material.

In some configurations of blends of the invention, the at least one oil is selected from triglycerides, fatty acids, fatty alcohols, fatty esters, vegetable oils and naturally occurring waxes and paraffins.

In some configurations of blends of the invention, the at least one oil is selected from mono-, di- and triglycerides, fatty acids, fatty alcohols, fatty esters, paraffins, iodinated oils, modified oils, fully synthetic oils, hydrogenated vegetable oils and waxes.

In some configurations of blends of the invention, the vegetable oil is selected from avocado oil, Brazil nut oil, Canola oil, coconut oil, corn oil, cottonseed oil, linseed oil, grape seed oil, hemp seed oil, olive oil, palm oil, peanut oil, poppyseed oil, sesame oil, soybean oil, walnut oil, sunflower oil, and cardanol oil.

In some configurations of blends of the invention, the vegetable oil is selected from castor oil, olive oil, soybean oil, sesame oil, palm oil, cardanol oil and poppyseed oil. In some configurations of blends of the invention, the at least one oil is an iodinated oil.

In some configurations of blends of the invention, the iodinated oil is an iodinated vegetable oil.

In some configurations of blends of the invention, the iodinated vegetable oil is iodinated poppyseed oil.

In some configurations of blends of the invention, the at least one oil is an ethyl ester of a fatty acid.

In some configurations of blends of the invention, the at least one oil is an ethyl monoiodostearate or ethyl diiodostearate.

In some configurations of blends of the invention, the at least one oil is a fatty acid or a fatty alcohol or a fatty ester having a fatty saturated or unsaturated acid or alcohol carbon chain, or both, wherein the saturated or unsaturated acid or alcohol carbon chain having between 8 and 18 carbon atoms.

In some configurations of blends of the invention, the fatty acid is selected from caprylic acid, capric acid, lauric acid, myristic acid, palmitic acid, stearic acid, myristoleic acid, palmitoleic acid, sapienic acid, oleic acid, elaidic acid, vaccenic acid, linoleic acid, linoelaidic acid, and α-linolenic acid.

In some configurations of blends of the invention, the fatty alcohol is selected from capryl alcohol, pelargonic alcohol, capric alcohol, hendecanol, lauryl alcohol, isotridecanol, myristyl alcohol, pentadecyl alcohol, cetyl alcohol, palmitoleyl alcohol, heptadecanol, stearyl alcohol, and oleyl alcohol.

In some configurations of blends of the invention, the fatty ester is selected from monoglycerides, diglycerides, and triglycerides of a fatty acid and/or a fatty alcohol.

In some configurations of blends of the invention, the at least one oil is a medium chain triglycerides (MCT).

In some configurations of blends of the invention, the MCT is formed of a medium chain fatty acid or a fatty alcohol having an aliphatic chain of 6 to 12 carbon atoms.

In some configurations of blends of the invention, the at least one oil is a hydrogenated fatty acid, fatty alcohol or fatty ester.

In some configurations of blends of the invention, the at least one oil in present in the blend in an amount between 10 and 60 wt %, between 10 and 50 wt %, between 10 and 40 wt %, between 10 and 30 wt %, between 10 and 20 wt %, between 20 and 50 wt %, between 20 and 40 wt %, between 20 and 30 wt %, between 30 and 50 wt %, between 30 and 40 wt %, or between 40 and 50 wt %.

In some configurations of blends of the invention, the blend is a homogeneous solid blend of at least one oil and at least one aliphatic polyester, wherein the blend having a mechanical and/or a physical property substantially identical to the same mechanical and/or physical property of the at least one aliphatic polyester alone, and wherein the at least one oil is selected from naturally derived oil optionally selected from triglycerides, fatty acids, fatty alcohols, fatty esters, vegetable oils and naturally occurring waxes; or synthetic or semisynthetic oils optionally selected from triglycerides, fatty acids, fatty alcohols, fatty esters, paraffins, iodinated oils, modified oils, fully synthetic oils, hydrogenated vegetable oils and waxes.

In some configurations of blends of the invention, the at least one oil has a melting temperature below 50° C.

In some configurations of blends of the invention, the mass ratio polymer to the oil (polymer: oil) is between 9:1 and 4:6.

In some configurations of blends of the invention, the oil content in the polymer blend is between 10 and 40% w/w.

In some configurations of blends of the invention, the blend may be in a form selected from nanoparticles, microparticles, films, fibers, slabs, sheets, rods, plates, filaments, and shape memory compositions.

In some configurations of blends of the invention, the blend comprising further at least one additive.

In some configurations of blends of the invention, the additive is selected amongst additives useful in treatment, prevention or diagnosis of a human body or amongst such additives capable of modifying at least one mechanical or physical attribute of the product.

In some configurations of blends of the invention, the additive is a non-active agent.

In some configurations of blends of the invention, the additive is a drug, an agriculturally active agent, a contrast agent, a metal oxide, a colorant, a binder, a solubilizing agent, a disintegrant, a lyophilizing agent, a stabilizer, an adhesive material, a surfactant, a toxicity reducing agent, a heat conducting agent, a radiation responsive element, and/or a magnetic material.

In some configurations of blends of the invention, the additive is an active agent selected from a pharmaceutically acceptable agent, a biomolecule, a small molecule, biomolecules, oligonucleotides, genes, carbohydrates, angiogenic factors, cell cycle inhibitors, hormones, nucleotides, amino acids, sugars, lipids, stem cells, antibiotic agents, antimicrobial agents, antifungal agents, anti-inflammatory agents, antioxidative agents, anti-proliferative agents, and anticancer agents.

In some configurations of blends of the invention, the additive is mixed within the polymer-oil blend or provided encapsulated in nanoparticles, nanocapsules, microparticles, microcapsules, liposomes, micelles, vesicles, or in a material delivery vehicle.

In some configurations of blends of the invention, the blend comprising nanoparticles or microparticles or capsules comprising at least one additive.

In some configurations of blends of the invention, the blend comprising at least one additive in a non-capsulated form.

In some configurations of blends of the invention, the blend comprises:
a hydrophilic drug in an encapsulated form;
a hydrophilic drug in an unencapsulated form;
a hydrophobic drug;
a hydrophobic drug in an encapsulated form;
a mixture of any of the above.

In some configurations of blends of the invention, the blend is configured for use as an implantable drug depot.

In some configurations of blends of the invention, the blend may be for use as a medical or cosmetic implant provided to replace or support or enhance an existing biological structure.

In some configurations of blends of the invention, the implant is configured for implanting under a subject's skin or within a body natural cavity or a cavity formed during a surgical procedure.

In some configurations of blends of the invention, the implant is a fiducial marker.

A fiducial marker formed of a blend according to any blend of the invention.

A fiducial marker comprising or consisting A homogeneous solid blend of at least one oil and at least one polymer, the blend having at least one mechanical and/or physical property substantially identical to the mechanical and/or physical properties of the at least one polymer alone, wherein the blend comprising between 10 and 60 wt % of the at least one oil and optionally one or more active or non-active additives.

In some configurations of markers of the invention, the marker is a biopsy marker or clip.

In some configurations of markers of the invention, the at least one oil is an iodinated oil.

An implant for therapeutic, diagnostic or cosmetic use, the implant formed of or comprising a homogeneous solid blend of at least one oil and at least one aliphatic polyester, wherein the blend having mechanical and/or physical properties substantially identical to the same mechanical and/or physical properties of the at least one aliphatic polyester alone, and wherein the at least one oil is a naturally derived oil optionally selected from triglycerides, fatty acids, fatty alcohols, fatty esters, vegetable oils and naturally occurring waxes; or a synthetic or a semisynthetic oil optionally selected from triglycerides, fatty acids, fatty alcohols, fatty esters, paraffins, iodinated oils, modified oils, fully synthetic oils, hydrogenated vegetable oils and waxes.

In some configurations of implants of the invention, the implant is a biopsy clip or a biopsy marker.

In some configurations of implants of the invention, the at least one polymer is at least one aliphatic polyester and wherein the at least one oil is lipiodol or iodinated poppy seed oil.

In some configurations of implants of the invention, the implant is in the form of a rod or film or a microsphere or nanoparticles formed of or comprising a homogeneous solid blend of at least one oil and at least one polymer, wherein the blend having a mechanical and/or physical property substantially identical to the same mechanical and/or physical property of the at least one polymer alone;

wherein the at least one oil is a naturally derived oil optionally selected from triglycerides, fatty acids, fatty alcohols, fatty esters, vegetable oils and naturally occurring waxes; or a synthetic or a semisynthetic oil optionally selected from triglycerides, fatty acids, fatty alcohols, fatty esters, paraffins, iodinated oils, modified oils, fully synthetic oils, hydrogenated vegetable oils and waxes; and wherein the at least one polymer is a homopolymer or a copolymer of lactic acid, glycolic acid, caprolactone and di- and tri-methylene carbonate.

A biopsy marker or clip formed of or comprising a blend according to the invention.

In some configurations of clips of the invention, the clip is configured for instillation in a subject's body by a needle or an installation device.

In some configurations of clips of the invention, for marking a site of a soft tissue.

In some configurations of clips of the invention, the soft tissue is a breast tissue or a tissue of the gastrointestinal tract.

In some configurations of clips of the invention, the tissue is marked during biopsy and imaged.

In some configurations of clips of the invention, the clip is suitable for imaging by mammography, CT, MRI or ultrasound.

In some configurations of clips of the invention, for placing in or in a vicinity of a soft tissue immediately following a biopsy procedure.

In some configurations of clips of the invention, when coated with a hydrogel or a mucoadhesive material.

In some configurations of clips of the invention, the hydrogel is or comprises hyaluronic acid, collagen, or polyethylene glycol.

In some configurations of clips of the invention, the clip is configured for instilling in a subject's body for a period of 1 or more month, 3 or more months, or 6 or more months, or 12 or more months.

In some configurations of clips of the invention, comprising one or more active agents.

In some configurations of clips of the invention, the active agent is a drug and/or a contrast agent.

In some configurations of clips of the invention, the contrast agent is an iodinated oil.

In some configurations of clips of the invention, the iodinated oil is an iodinated vegetable oil.

In some configurations of clips of the invention, the iodinated oil is iodinated poppyseed oil.

In some configurations of clips of the invention, the contrast agent is an ethyl monoiodostearate or ethyl diiodostearate.

In some configurations of clips of the invention, the contrast agent is diatrizoate, metrizoate, iothalamate, ioxaglate, iopamidol, iohexol, ioxilan, iopromide, iodixanol, iobitridol, ioversol, gadoterate, gadobutrol, gadoteridol, gadopentetate, gadobenate, gadopentetic acid dimeglumine, gadoxentate, gadoversetamide, or gadodiamide.

A clip or a biopsy marker formed of or comprising a homogeneous solid blend of at least one oil and at least one aliphatic polyester, wherein the blend having a melting temperature substantially identical to the melting temperature of the aliphatic polyester alone;
wherein the at least one oil is a naturally derived oil selected from triglycerides, fatty acids, fatty alcohols, fatty esters, vegetable oils and naturally occurring waxes; or a synthetic or a semisynthetic oil selected from triglycerides, fatty acids, fatty alcohols, fatty esters, paraffins, iodinated oils, modified oils, fully synthetic oils, hydrogenated vegetable oils and waxes; and wherein
the at least one polymer is a homopolymer or a copolymer of lactic acid, glycolic acid, caprolactone and di- and tri-methylene carbonate.

A process for manufacturing a solid blend according to the invention, the process comprising treating a mixture of at least one oil and at least one polymer, under shear forces, optionally in the presence of a solvent, at a temperature between room temperature (25-30° C.) and a melting temperature of the at least one polymer.

In some configurations of processes of the invention, the melting temperature of the at least one polymer is between 5° and 200° C.

In some configurations of processes of the invention, the shear forces applied are achievable by blending, mixing, homogenization, or vortexing.

In some configurations of processes of the invention, the process comprising treating a mixture of at least one oil and at least one polymer, in the absence of a solvent, at a temperature between room temperature and the melting temperature of the at least one polymer, wherein the treating comprises application of shear forces to the mixture until a homogenous blend is obtained, and cooling the blend to obtain the solid blend.

In some configurations of processes of the invention, the process comprising treating a mixture of at least one oil and at least one polymer, in presence of a solvent, at a temperature between room temperature and the melting temperature of the at least one polymer, wherein the treating comprises application of shear forces to the mixture until complete or substantially complete evaporation of the solvent and formation of a homogenous blend, and cooling the blend to obtain the solid blend.

In some configurations of processes of the invention, treating comprises homogenizing the mixture.

In some configurations of processes of the invention, treating comprises homogenizing the mixture followed by continuous stirring for a time period sufficient to cause solvent evaporation.

In some configurations of processes of the invention, when a solvent is used, the solvent is a volatile organic solvent.

A solid homogeneous blend of at least one aliphatic polyester and at least one oil, the blend formed by applying a shear force to a mixture of the at least one aliphatic polyester and the at least one oil until a single-phase blend material is obtained, wherein the blend exhibits a melting temperature that is substantially same as the melting temperature of the at least one aliphatic polyester.

In some configurations of blends of the invention, the blend is a bioplastic configured for use in a method of manufacturing a biodegradable object.

In some configurations of blends of the invention, the object is selected from disposable syringes and medical supplies, boards, coatings, rods, 3D printing inks, containers, trays, sheets, foams and packaging materials.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 provides images of films of PLA with various amounts of sesame oil. All films are flexible solids with no surface oil feel.

FIGS. 2A-B provide FTIR spectra of different locations (FIG. 2A) on a PLA-sesame oil film that were analyzed. The FTIR absorbance at ˜2923 and ˜2854 cm⁻¹ absorbance for the sesame oil in the entire film indicate that the oil blends very homogeneously with the polymer too (FIG. 2B).

FIG. 3 shows fluorescence confocal analysis (x100) of PLA-sesame oil films in the presence of Nile red. It should be noted that with increasing oil content in the films, the hydrophobicity increases proportionally for the Nile red, resulting in increasing fluorescence intensity.

FIG. 4 provides SEM analysis of vertical cross-section of PLA-sesame oil films. All films were prepared in the similar way as disclosed herein.

FIG. 5 provides SEM analysis of surface regions of PLA-sesame oil films facing air (U) and facing a mold surface (D).

FIG. 6 provides SEM analysis of a surface of a PLA-sesame oil film with 50% oil content. The average size of the oil filled bulbs in the films is 2-3 μm.

FIGS. 7A-B provide non-isothermal DSC scan of PLA in the presence of sesame oil. (FIG. 7A) First thermal scan, (FIG. 7B) Second thermal scan after non-isothermal cooling. “Company PLA” indicate untreated PLA (the PLA obtained directly from the manufacturer), and “0-50%” means PLA films prepared using chloroform.

FIG. 8 depicts the release of p-nitrophenol from the PLA-sesame oil films. The amount of p-nitrophenol in the films was 10% (w/w).

FIG. 9 demonstrates the foldability of PLA-sesame oil films with 50% (w/w) oil. The films remained foldable after 110 days of hydrolytic degradation. Similar observation was experienced with PLA films with 40% (w/w) sesame oil content.

FIG. 10 depicts the % weight loss in PLA-sesame oil films after hydrolytic degradation. The inset represents the amount of sesame oil content (w/w) in the films.

FIGS. 11A-J provides SEM analysis of a surface of PLA-sesame oil films. For 0% oil content; (FIG. 11A) and (FIG. 11B) are the two sides of the films, (FIG. 11C) is the vertical cross-section and (FIG. 11D) is the interior of the film. For 20% oil content; (FIG. 11E) and (FIG. 11F) are the two sides of the films, (FIG. 11G) is the vertical cross-section and (FIG. 11H) is the interior of the film. For 50% oil content; (FIG. 11I) is the vertical cross-section and (FIG. 11J) is the interior of the film.

FIG. 12 (i-left image) provides a representative UV absorption of ESC in 10 mM PBS pH 7.2, (ii-middle image) is the ESC standard curve, and (iii-right image) is a representative picture for the microparticles dry powder.

FIG. 13 provides SEM images for the microparticles prepared with PLA. Samples were labelled as follows: (i) PLA-MCT oil, (ii) PLA-sesame oil, and (iii) PLA-castor oil.

FIG. 14 provides SEM images for blank PLA microparticles were (i) made with PLA only, (ii) made with 1:1 PLA: castor oil. The images show that the particles were condensed to form biconcave disk like shape in the presence of oil.

FIG. 15 provides in vitro accumulative release graph for escitalopram (ESC) in PBS pH 7.2 for the developed microparticles prepared with (i) PLA and (ii) PLGA. The oil and ESC content in the preparation step were 40 and 20% w/w respectively. Samples were analyzed in duplicate.

FIG. 16 provides SEM images for microparticles prepared with PLA. Samples were labelled as follows: (i) PLA-coconut, (ii) PLA-cetyl alcohol, (iii) PLA-stearic acid. The polymer to oil ratio was 1:1.

FIG. 17 provides SEM images for microparticles prepared with PLA. Samples were labelled as follows: (i) PLA-40% sesame, (ii) PLA-30% sesame, (iii) PLA-PLA-20% sesame, and (iv) PLA-10% sesame.

FIG. 18 provides in vitro accumulative release graph for ESC in PBS pH 7.2 for the developed microparticles prepared with (i) PLA with different ratio of sesame oil and (ii) PLA with coconut, cetyl alcohol and stearic acid. Samples were analyzed in duplicate.

FIG. 19 provides DSC scan of PCL blends.

### DETAILED DESCRIPTION OF EMBODIMENTS

### Example 1: PLA-Lipiodol for Biodegradable Clip Markers

Three polymers abbreviated Polymer A (Poly(lactic-co-glycolic acid) (50:50); Polymer B (PLGA (Poly(lactic-co-glycolic acid)) (75:25); and Polymer C (Polycaprolactone (PCL)) mixed with 20% Lipiodol and 0.2% iron-oxide nanoparticles; and a control polymer were implanted into nine mice, followed with CT and MRI imaging. Specimens were examined for tissue analysis of iodine and iron content. Significant differences in polymer resorption and visualization on CT were noted particularly at 8 weeks (p<0.027). Polymers A, B, and C were visible by CT at 4, 6, and 8 weeks, respectively. All marker locations were detected on MRI (T1 and SWI) after 24 weeks, with tattooing of the surrounding soft tissue by iron deposits. CT and MR visible polymers markers can be constructed to possess variable resorption with stability ranging between 4- and 14-weeks post placement, making this approach suitable for distinct clinical scenarios with varying timetables.

### Materials and Methods

Based upon clinical need, the compounds for evaluation needed to achieve CT and MR conspicuity lasting between 4 and 24 weeks, to cover a wide range of scenarios.

Materials: Materials used for the preparation of lipiodol implants included: PCL (Polycaprolactone); Mw 14000 Da and Iron (II,III) Oxide nanopowder 50-100 nm particle size (Lot #MKBR5062V) (Sigma Aldrich, Israel); PLGA (Poly(lactic-co-glycolic acid)) 50:50 (Lactic acid to Glycolic acid ratio); Mw 17 kDa and PLGA 75:25; Mw 18 kDa (PURAC, The Netherlands); Lipiodol Ultra Fluid, Iodine content 0.49 mg/mL Ch-B: 17LU602A (Guerbet, Villepinte, France), and Cloroform (CHCI3) (BioLab, Israel).

### Polymer Marker Preparation

Each of the three polymers used in this study contained 20% Lipiodol and 0.2% w/w iron oxide. The three polymer compounds were composed of: Polymer A-PLGA 50:50; Polymer B-PLGA 75:25, and Polymer C-PCL. The polymers were formed into a rod shape of an adequate 1 mm diameter to fit into a biopsy syringe, similar to other commercially metallic markers used in clinical practice. Lipiodol, iron oxide, and polymer were added to a 5 mL glass vial and the mixture was heated to 80° C. for 30 mins while hand mixing with a spatula to form a viscous liquid. The molten mixture was then transferred to a hot glass syringe connected to a 17 G needle and casted by pressing through the needle to form black cylindrical rods. The rods were then cut into 2-3 mm implants using an 11-blade scalpel and used for further studies.

### Murine In-Vivo Model

A total of 9 male BaLB/C OLAHSD mice, 8-9 weeks of age, weighing approximately 20 g were obtained from Harlan Laboratories (Rehovot, Israel). Each mouse had four markers inserted, the three study polymers and a control substance (the polymer marker without lipiodol or iron). Mice were housed in cages with free access to food and water. Animal care and the test injections were conducted at a good laboratory practice (GLP) certified site (Sharett institute SPF unit, Hadassah Medical School), in accordance with the National Institutes of Health guide for the care and use of Laboratory animals. The animals were anesthetized with Ketamine-Xylazine cocktail: 87.5 mg/kg ketamine (Ketaset, 100 mg/mL, Fort Dodge, Iowa, USA) and 12.5 mg/kg xylazine (20 mg/mL, Biob, France) administrated Intramuscular (IM) at a dose of 5 mL/kg bodyweight. Mice were anesthetized and underwent subcutaneous insertion of polymer markers using a coaxial needle technique (17-gauge) to the back (right and left upper and lower regions, respectively). Three 3 mm×1 mm markers containing the polymer formulas (A, B, and C) and one control marker, PSA: RA polymer (polymer without contrast agents), were inserted. The location of each polymer marker type was random for each mouse. Euthanasia was achieved by means of Carbon dioxide, according to institutional animal care and use committee guidelines.

Imaging was performed at baseline 24 hours after implantation and at two week scheduled intervals to 6 months (13 scans per mouse). Imaging protocols included serial CT and a final MRI.

CT was performed on anesthetized mice, in the prone position, imaged on a 64 detector scanner (Brilliance 64 CT scanner, Philips Medical Systems, Cleveland, OH). Scans were performed with the following parameters: 120 kV, 70 mAs, collimation 64×0.625 slice thickness 0.9 mm, increment 0.45 mm rotation time 0.5 s and pitch of 0.641. Images were reconstructed, using bone and soft tissue algorithms. Follow up scans were performed every 2 weeks, up to 24 weeks.

MR imaging was performed at 6 months for all implanted mice immediately after sacrifice. Mice were placed in the prone position and imaged using a 1.5T clinical scanner (Avanto, Siemens Healthcare, Belgium) with a 16-channel body coil placed above the animals. Based on prior experience, two relevant sequences were acquired: T1 (TR=2300 ms, TE=3.05 ms) weighted images with 256 mm field of view with a 265*265 matrix, a section thickness of 1 mm with 7.3 mm spacing and Susceptibility Weighted Images (SWI), with a 230*230 mm matrix on a 230 mm field of view.

Radiologic/Pathologic Evaluation: Radiologic-pathologic correlation was performed following mice sacrifice at 6 months. Radiology images of all scans for all mice were reviewed for conspicuity by three readers (SNG, EBD and HL) in consensus and then subsequently compared over time on a mouse-by-mouse and marker-by-marker basis. For purpose of evaluating clinically acceptable conspicuity of the polymer markers, a five-point scale was used to determine visualization quality of the markers and the degree of resorption, until deemed not useful clinically. The categories of the scale were: 1-baseline (post insertion), 2-mild resorption (clearly visible), 3-substantial resorption (<50% of initial marker), 4-near total elimination (barely visible), and 5-total elimination. The baseline, mild and substantial resorption were regarded as clinically acceptable, the fourth and fifth (near total elimination and total elimination) were not considered to be adequate for clinical use.

Post-sacrifice, gross and histopathology specimens were extracted by resecting the region of the marker placement. The specimens were stained with hematoxylin-eosin. All tissue specimens were examined and evaluated for polymer degradation, iron retention, and inflammatory response.

Representative remaining identified material in the region of marker implantation was sent for biochemical analysis including an Iodine/Iron analysis by four separate analyses. This included Energy Dispersive X-Ray Analysis (EDX), an X-ray technique used to identify the elemental composition of the materials. The EDX instrument with Scanning Electron Microscopy (Quanta 200, FEI Company) was equipped with an EDAX detector. To prevent burning, specimens were sputter coated with palladium at 40 mV for 40 s prior to analysis. Scanning electron microscopy (SEM) (VEGA3, TESCAN) was used for imaging and analysing the size and morphology of specimens. The specimens were visualized under vacuum (upper limit of 6×10−6 mbar) and images were taken with beam excitation energy of 20 kV. Next, Fourier Transform Infrared Spectroscopy (FTIR) spectra were recorded with a Thermo Scientific FTIR spectrometer (Smart iTR Nicolet iS10 FT-IR) with diamond crystal. A 5-10 mg sample was placed into a crystal window and the spectrum recorded. The scanning range was 400-4000 cm⁻¹ with a resolution of 4 cm⁻¹. The number of scans for each sample was set to 10. Finally, Nuclear magnetic resonance spectroscopy (1H NMR) was performed using a Varian Mercury 300 MHz NMR spectrometer. A 10 mg polymer sample was dissolved in 2 mL of deuterated chloroform (CDCl3). The sample solution was transferred to NMR tubes of 5 mm diameter.

### Statistics

For each arm, results were analyzed for each time point comparing the resorption rates for each marker in each mouse, using Multi-variate ANOVA with T-tests performed for various time points if p<0.05. Additionally, multiple Kaplan Meier plots with a 95% CI were constructed using the transition between the different grades as the defining endpoint event using MedCalc® Statistical Software version 19.5.3 (MedCalc Software Ltd, Ostend, Belgium) for analysis.

### Results and Discussion

CT Imaging: Eight mice survived to the 6-month study endpoint, with one mouse expiring at week 16 upon anesthetic injection (autopsy showing no pathologic abnormalities or signs of infection or excessive inflammation). Conspicuity on CT, based on the grading system described in the methods section, are summarized in Table 1. All polymers had similar maximal conspicuity at baseline and showed evidence of degradation over the 6-month study period. However, significant differences in the rate of degradation were observed among the polymers overall (ANOVA; P<0.01), particularly at 8 weeks (p<0.027). Overall, polymer A began to demonstrate diminution of conspicuity at 4 weeks. However, all markers with this compound remained sufficiently perceivable (grades 1-3) for six weeks. Subsequently, a decline to inadequate levels of visualization (grades 4-5) was seen in 44% by 14 weeks/Polymer B remained with excellent conspicuity for at least 8 weeks (grade 1-2), with all animals demonstrating clinically relevant (grades 1-3) for up to 12 weeks. Thereafter, a rapid decline in conspicuity was noted, rendering the markers not clinically usable. Polymer C demonstrated rapid loss of contrast visualization by the 4th week. At six months, there was no visibility of Polymer A in all animals, borderline visibility (grade 3) in 33% of Polymer B, and 11% in Polymer C. As anticipated, the control polymer was not visualized on CT discreetly from the subcutaneous tissues.

TABLE 1
Grading of degree of contrast resorption on CT for the three polymers (numbers represent percentages)

| | | | | | Time post implantation | | | | | |
|---|---|---|---|---|---|---|---|---|---|---|
| | | | 4 | 6 | 8 | 10 | 12 | 14 | 16 | 24 |
| | Resorption | Day 1 | weeks | weeks | weeks | weeks | weeks | weeks | weeks | weeks |
| Polymer A | 1-Baseline | 100 | 56 | 44 | 22 | 0 | 0 | 0 | 0 | 0 |
| (PLGA | 2-Mild | 0 | 22 | 33 | 33 | 56 | 11 | 11 | 0 | 0 |
| 50:50) | 3-Substantial | 0 | 22 | 22 | 33 | 22 | 67 | 44 | 56 | 0 |
| | 4-Near total | 0 | 0 | 0 | 11 | 11 | 0 | 22 | 11 | 56 |
| | 5-Total | 0 | 0 | 0 | 0 | 11 | 22 | 22 | 33 | 33 |
| Polymer B | 1-Baseline | 100 | 89 | 22 | 11 | 0 | 0 | 0 | 0 | 0 |
| (PLGA | 2-Mild | 0 | 11 | 78 | 89 | 67 | 33 | 0 | 0 | 0 |
| 75:25) | 3-Substantial | 0 | 0 | 0 | 0 | 33 | 67 | 56 | 44 | 33 |
| | 4-Near total | 0 | 0 | 0 | 0 | 0 | 0 | 44 | 33 | 0 |
| | 5-Total | 0 | 0 | 0 | 0 | 0 | 0 | 0 | 22 | 56 |
| Polymer C | 1-Baseline | 100 | 22 | 22 | 22 | 0 | 0 | 0 | 0 | 0 |
| (PCL) | 2-Mild | 0 | 44 | 44 | 33 | 44 | 22 | 11 | 11 | 0 |
| | 3-Substantial | 0 | 11 | 0 | 11 | 11 | 22 | 0 | 0 | 11 |
| | 4-Near total | 0 | 11 | 0 | 0 | 0 | 11 | 22 | 22 | 11 |
| | 5-Total | 0 | 11 | 33 | 33 | 44 | 44 | 67 | 67 | 67 |

Kaplan Meier plots for the three polymers was significant with a 95% CI (p<0.0001, x2=23.25) for resorption from grade 3 (substantial resorption—but still clinically visible) to grade 4 (trace visualization-deemed challenging for good visualization in clinical practice) and additionally from grade 4 to grade 5 (trace visualization vs. total resorption) (p<0.0001, x2=32.58). Transition from grades 1-2 and 2-3 were not statistically significant (p>0.05).

MR imaging: At 24 weeks, there was susceptibility signal at the injection site on T1 and to an even greater degree on the SWI images, without significant distortion of the image at all insertion sites, even in mice where there was total elimination of conspicuity on CT. No MR signal was detected at the injection site of the control marker.

Radiologic-pathologic correlation: Gross pathologic inspection demonstrated marked dissolving of the markers in all cases where no lipiodol was detected at CT. Two-three mm residual “blobs” were identified for those 4 injection sites with residual CT signal. In the remaining 20 injection sites with total or near total elimination, there was 1-2 mm region of a blackened tattoo-like appearance of the soft tissues surrounding the initial implant site which contained clusters of highly pigmented macrophages. Histopathologically, the insertion site was surrounded by several layers of fibroblasts, representing a characteristic inflammatory response. Control injection sites had no pockets of macrophages and only limited inflammatory reaction adjacent to fat and muscle.

The size of the markers that were obtained after 6 months were small and weighed ˜1 mg for the Polymer A and Polymer B markers, whereas the Polymer C markers weighed 3-5 mg. SEM for iron nanoparticles demonstrated scattered clusters of iron nanoparticles over the surface of the markers. The detection of the iron content by EDX was not possible for any of the markers. Since, the amount of the marker clips recovered after 6 months in vivo studies was very low, thus, the total iron content in the recovered materials were below the minimal detectable elemental concentration. On the other hand, iodine was detected in Polymer A and Polymer B markers, whereas the Polymer C marker did not show any sign of iodine.

PLGA 75:25 (Polymer B) polymer markers, not depicted here, demonstrated a profile similar to Polymer A.

Proton (1H) nuclear MR spectroscopy measurements were performed to identify chemical structures in lipiodol. It should be noted that lipiodol is not a pure compound and hence, its exact chemical structure is still unknown. Lipiodol® is an iodinated (480 mg iodine/ml) and ethylated ester derived from poppy seed oil. However, the signals in its 1H spectrum can be used to identify its presence in the polymer clips. The characteristic peaks at 0.96 ppm, 2.25 ppm and 4.27 ppm can be used to distinguish lipiodol from the polymers in the clip formulation. NMR analysis of the markers also confirmed the presence of lipiodol remaining in the samples after 6 months. However, 1H NMR splitting of the polymer and lipiodol could not be determined in the marker samples because of excess dissolved tissue samples along with iron oxide nanoparticles. Further, for Polymer C markers, no NMR splitting at 0.96 ppm was observed. For Polymer A markers, no characteristic NMR signals were detected, whereas Polymer B markers showed weak, but characteristic peaks of lipiodol at 0.96 ppm.

Finally, FTIR also detected the presence of lipiodol in the Polymer B markers. Absorption bands at ˜1744 cm⁻¹ is assigned for C═O stretching which is similar to the blank PLGA clips. The FTIR spectra of lipiodol will resemble the FTIR of the poppy seed oil. Like polymers, the regions of 1700-1800 cm⁻¹ for the C—H stretching existed in the lipiodol. The visible characteristic absorbance that distinguishes lipiodol from polymer spectra is only possible at 2854 cm⁻¹ that is due to symmetric C—H from methylene chains and the band at ˜1371 cm⁻¹ for the O—CH₂ groups,

In this study, three degradable polymers formulated and designed to be used as implantable markers visible by multi-modality imaging in an in-vivo model and note substantially different rates of degradation for different formulations were investigated. Polymer B (PLGA 75:25) achieved the slowest resorption and generated the longest acceptable conspicuity on CT—at least until week 12 post insertion. Hence, it is likely the most suitable for clinical scenarios such as radiation therapy where a 3-month period of visualization on CT and MRI may be considered ideal. Radiation planning for most tumors (such as breast and liver cancer) falls within the 12-week window. This conspicuity may also most beneficial for CT guided procedures, such as thermal and non-thermal ablation of tumor and image guided biopsies. While the extended 12-week conspicuity achieved with polymer B may be beneficial in some clinical instances, some, such as breast surgery, may favor a shorter, 6-week, degradation period, as attained with polymer A. Yet, the short-lived conspicuity of polymer C may render it practical only for procedures requiring immediate use (i.e. within days of the insertion).

It is important to note that even after CT conspicuity is lost, MR conspicuity was maintained even at 6 months, likely due to macrophage iron retention. This is also evident on the gross pathology specimens as a tattooing phenomenon and the scanning electron microscopy results, apparently independent of polymer iodine content. Furthermore, regarding retention of iodine, whereas in polymers A and B the iodine-containing-lipiodol bonded, albeit with decrease in marker size and reabsorption over time. To the contrary, polymer C is unable to hold the lipiodol and thus, the oily lipiodol leaks out over time from the polymer matrix. This was observed in our study as a decline in the contrast visualization of Polymer C markers on CT by week four post implantation.

In addition to the polymer composition, the payload may also be hypothetically tailored for specific clinical indications. If a marker is needed to be visible for a long-term medical situation and specifically requires visualization on MRI, then the desired polymer may contain an iron compound. Additionally, iron is beneficial because frank metallic markers will generally have a large artefact on MRI, but theoretically the amount of iron can be altered to allow MRI visualization while minimizing susceptibility artefact. Indeed, iron's effects are much less pronounced on T1 gradient echo sequences than on SWI images. Thus, if needed, radiation planning may be performed based on MR images, using T1 and SWI sequences. However, if the requirement is for a short-term condition and for CT only, a polymer containing only lipiodol may likely be sufficient.

The histopathologic examination showed dissolution of the polymer marker, with minimal expected inflammatory response, thus adding to the accumulating data on the safety profile of the polymers. Control sites did not demonstrate any significant finding beyond minimal inflammatory changes. Thus, it is likely that the iron deposits seen within macrophages at the insertion site, although they contribute to the visualization on MRI even after 24 weeks, are responsible for the mild inflammation.

Preparation of Clips with Different Shapes for Distinguishing Among Implanted Clips.

To achieve mammography clips with different shapes while implanting a rod shape clips, Shape Memory Polymers were used. Alternatively, rods were designed to have different Lipiodol content along the implanted rod. To obtain rods with certain parts with Lipiodol and other parts are Lipiodol free, coextrusion of the polymer and the polymer/lipiodol is applied to obtain clips with different visibilities. Also, 3D printing can be used to prepare shape memory polymers.

Shape memory clips: PLA based shape memory polymer was prepared following the method of Langer and Lendlein, U.S. Pat. No. 6,160,084. In brief, low-molecular-weight poly(lactide-co-glycolide) diol (PLGA, Mn 2000 g/mol) by copolymerization of glycolide with L,L-lactide with ethylene glycol as an initiator. The copolymer diol was further connected with oligo (p-dioxanone)diol (ODX) with a diisocyanate linker to obtain multiblock polyester-urethane. The PLGA diol and ODX, the soft and hard segments, respectively, functioned as the shape-switching and shape-fixing parts of the polymer network. The polymer was mixed with lipiodol at a 30% loading along with 0.2% of iron oxide and casted into rods. Different shapes were designed so that after implantation in the marking site, the rod changes its shape due to exposure to tissue environment.

Hydrogel coating of biodegradable clips: In order to better visualize the clip by MRI and ultrasound, the biodegradable clip is coated with a thin coating of dry hydrogel material that upon contact in tissue absorbs water and swell. The swollen coating is water which is better visible by proton MRI and to some extent is better visualized by ultrasound. The in situ swelling also improves the fixation of the clip marker in the tissue site. The hydrogel coating should remain on the clip as long as it is required for the medical need for visualization. Hydrogel coatings should be biodegradable and fixed onto the clip. Biodegradable swellable hydrogel materials such as hyaluronic acid (HA), crosslinked dextran, oxidized cellulose or starch, chitosan, polyethylene glycol (PEG), copolymers of ethylene glycol and propylene glycol and copolymers of a biodegradable polyester and PEG such as PLGA-PEG. The clips of this invention are coated with for example, hyaluronic acid solution and crosslinked.

In a typical experiment, sodium salt of HA with an average molecular weight of 2.0 106 was used as dry powder. Glutaraldehyde (GA), and divinyl sulfone (DVS) were purchased from Sigma. In order to obtain a crosslinked HA gel with divinyl sulfone (DVS), HA was dissolved in dilute alkaline solution (4 wt %) and DVS was added dropwise. Generally, one hour was enough for completion of the crosslinking reaction. The clips were coated with this mixture by dipping in the gel solution and allowed to crosslink. All gels were optically clear, with a smooth surface. For crosslinking to proceed with GA the aqueous HA mixture should be acidic and varying amounts of crosslinker were used. This crosslinker produced the most mechanically robust gels and they were also easily moulded into any shapes. All gels were washed to remove any unreacted crosslinker before drying.

In another experiment, the clip is dipped in a solution of chitosan at pH 5.5 and the coated clips are dried or pass through a water pH7.4 to fix the chitosan onto the clip. The chitosan solution may contain a hydrogel or a polysaccharide.

### Example 1A: Polymer-Lipiodol Blends to Form Nanoparticles

Lipiodol polymer nanoparticles were prepared for use as injectable biodegradable contrast agent for diagnostic purposes. The visible nanoparticles by CT are coated with homing agents to target the contrast agent to certain cells or tissues such as cancerous cells or tissues. The nanoparticles may contain an anticancer drug to serve as theragnostic agent. The particles were prepared by antisolvent-emulsion based dispersion method. Nanoprecipitation method in organic medium is restricted, since lipiodol and other oils are soluble in most of the organic solvents. Poly(ε-caprolactone) (PCL) 14,000; Poly(lactide-glycolide) (PLGA) 50:50 and 75:25, MW=15,000; Lipiodol; poppyseed oil, lecithin; Polyvinyl alcohol (PVA, MW=30,000-70,000) and chloroform were used to make the nanoparticles. 20% lipiodol was incorporated in nanoparticles.

In a typical experiment, PCL (800 mg) (or PLGA), Lipiodol or poppyseed oil (200 mg) and lecithin or phospholipid bound to a fluorescent marker or a specific ligand (100-500 mg) were dissolved in 10 mL chloroform. Polyvinyl alcohol (PVA) aqueous solution (1% w/v) was prepared by dissolving 1 g in 100 mL water. The solution was then filtered using Whatman filter paper to remove undissolved material. The organic solution mixture was added to the aqueous phase and homogenized. Different speed and time were examined. The homogenized mixture was then kept on magnetic stirring (1,000 rpm) for 1-5 h, until the chloroform evaporated to form the nanoparticles. The samples were centrifuged at 13,500 rpm for 15 minutes at room temperature. The obtained pellets were re-dispersed in water and analyzed.

Results: The particle size of the nanoparticles is given in Table 2 for poppyseed oil and in Table 2 for Lipiodol

TABLE 2
DLS analysis of nanoparticles obtained following the protocol
mentioned above in the presence of poppyseed oil

| | organic | | | | | |
|---|---|---|---|---|---|---|
| | to aq. | | | | | Zeta |
| | Phase | Homogenizer | Homogenization | Size (nm) | | potential |
| No.* | ratio | speed (rpm) | time (min) | (dispersed) | PDI | (mV) |
| 1 | 1:5 | 19000 | 2 | 796 ± 93 | 0.631 ± 0.108 | — |
| 2 | 1:5 | 26000 | 1 | 482.4 ± 57 | 0.441 ± 0.177 | — |
| 3 | 1:5 | 26000 | 5 | 313.3 ± 6.7 | 0.230 ± 0.052 | 44.4 ± 0.4 |
| 4 | 1:2.5 | 26000 | 5 | 376.7 ± 3.4 | 0.199 ± 0.092 | 49.4 ± 0.1 |

*The samples were centrifuged, and the pellets were re-dispersed in water and analyzed for DLS

TABLE 3
DLS analysis of nanoparticles obtained following the
protocol mentioned above in the presence of lipiodol

| | organic | | Time of | | | Zeta |
|---|---|---|---|---|---|---|
| S. | vs aq. | Homogenizer | homogenization | Size (nm) | | potential |
| No.* | phase | speed (rpm) | (min) | (dispersed) | PDI | (mV) |
| 1 | 1:5 | 26000 | 3 | 397.9 ± 19.8 | 0.369 ± 0.164 | −48.8 ± 0.7 |
| 2 | 1:5 | 26000 | 3 | 268.2 ± 1.1 | 0.340 ± 0.054 | −48.7 ± 3.8 |

*The samples (no. 1, and 2) were centrifuged, and the pellets were re-dispersed in water and analyzed for DLS

H-NMR and FTIR analysis of the nanoparticles indicated full content of Lipiodol or poppyseed oil in the particles.

A 5 grams scale batch was prepared. The results are reproducible and there is no significant difference in the particle size from the small batch and large sample batches.

TABLE 4
DLS analysis of nanoparticles obtained following the
protocol mentioned above in the presence of lipiodol

| | organic | | Time of | | |
|---|---|---|---|---|---|
| | vs aq. | Homogenizer | homogenization | Size (nm) | |
| S. No.* | phase | speed (rpm) | (min) | (dispersed) | PDI |
| 1 (MA019) | 1:5 | 26000 | 3 | 268.2 ± 1.1 | 0.340 ± 0.054 |
| 2 (YK-1-33) | 1:5 | 26000 | 3 | 225.7 ± 14.5 | 0.342 ± 0.023 |

*The samples (no. 1, and 2) were centrifuged, and the pellets were re-dispersed in water and analyzed for DLS

### 2. PLGA (50:50)-Lipiodol NPs

The protocol as mentioned above for PCL was implemented directly on the PLGA (50:50)-Lipiodol material to obtain the NPs. PLGA (50:50) of 15,000 molecular weight was used.

TABLE 5
DLS analysis of nanoparticles obtained following the
protocol mentioned above in the presence of lipiodol

| | organic | | Time of | | | Zeta |
|---|---|---|---|---|---|---|
| | vs aq. | Homogenizer | homogenization | Size (nm) | | potential |
| No.* | phase | speed (rpm) | (min) | (dispersed) | PDI | (mV) |
| 1 | 1:5 | 26000 | 3 | 263.4 ± 3.6 | 0.336 ± 0.044 | −49.3 ± 3.5 |
| 2 | 1:5 | 26000 | 3 | 240.7 ± 1.8 | 0.256 ± 0.007 | −51.7 ± 3.9 |

*The samples (no. 1, and 2) were centrifuged, and the pellets were re-dispersed in water and analyzed for DLS

### 3. PLGA (75:25)-Lipiodol NPs

The protocol as mentioned above was implemented directly on the PLGA (75:25)-Lipiodol material to obtain the NPs. Mw of PLGA (75:25) is 15,000.

TABLE 6
DLS analysis of nanoparticles obtained following the
protocol mentioned above in the presence of lipiodol

| | organic | | Time of | | | Zeta |
|---|---|---|---|---|---|---|
| S. | vs aq. | Homogenizer | homogenization | Size (nm) | | potential |
| No.* | phase | speed (rpm) | (min) | (dispersed) | PDI | (mV) |
| 1 | 1:5 | 26000 | 3 | 190.3 ± 3.3 | 0.206 ± 0.008 | −48.4 ± 5.1 |
| 2 | 1:5 | 26000 | 3 | 241.9 ± 1.2 | 0.244 ± 0.008 | −43.0 ± 0.9 |

*The samples (no. 1, and 2) were centrifuged, and the pellets were re-dispersed in water and analyzed for DLS

These nanoparticles were visible when visualized by CT.

### Example 2: Biodegradable Blends of Polylactic Acid and Sesame Oil

This example describes the effect of blending of non-modified vegetable oils on the properties of the PLA. Herein, a solvent casting method for the preparation of the PLA-oil blend and the controlled release of the model active agent from the PLA-oil thin films was studied. The changes in the physical properties of the PLA using a wide range of oil concentration between 0-50% by weight in the polymer blend.

### Materials and Method

Materials: Polylactic acid (PLA) (Mw100 kDa); Sesame oil; Chloroform; Nitrophenol. Method: A total material of polymer oil blend of 1 gram includes an sesame oil percentage of 10-50% w/w. Both sesame oil and PLA were weighed in the glass vials and 10 ml of chloroform was added to the blend. The sample was mixed over vortex for overnight at room temperature. A clear solution was obtained, and the mixture was kept drying at room temperature to evaporate the organic solvent in silicon molds covering with big beakers under closed hoods. This process helps in the removal of air bubbles that could formed due to condensation.

### Instrumentation

Scanning electron microscopy (SEM): SEM samples were sputtered by Pd/Au (SC7620, Spatter coater, UK), and examined with FEI Quanta 200 SEM (Brno, Czech Republic).

Fourier transform infrared spectroscopy (FTIR): FTIR spectra were recorded with a Thermo Scientific FTIR spectrometer (Smart iTR Nicolet iS10 FT-IR) with diamond crystal. The instrument requires ˜5-10 mg sample that is placed onto a crystal window and spectrum recorded. The scanning range was 400-4000 cm⁻¹ and the resolution was 4 cm⁻¹. The number of scans for each sample was set to 32.

Differential Scanning calorimetry (DSC): The thermal properties of polymer-oil blend were measured by a DSC-1, Mettler Toledo instrument under a nitrogen atmosphere. Each sample of about 8 mg in an aluminium crucible was heated from 25 to 200° C. at a scan rate of 10° C./min. Each sample was run in triplicate. The results are presented as the average data.

Confocal fluorescence analysis: Spinning disc confocal microscope (Nikon) was used for the fluorescence confocal analysis (x100) of the polymer-oil films in the presence of Nile red.

Elongation strength measurement: Instron material tester was used to analyze the elongation strength of the polymer-oil films. Dog-bone-shaped specimens were cut from the film samples with a sharp single-edge razor or scalpel and further analyzed using the machine.

Benchtop NMR Measurements: Measurements were performed on a Bruker Minispec 20 at 37° C.

### Results and Discussion

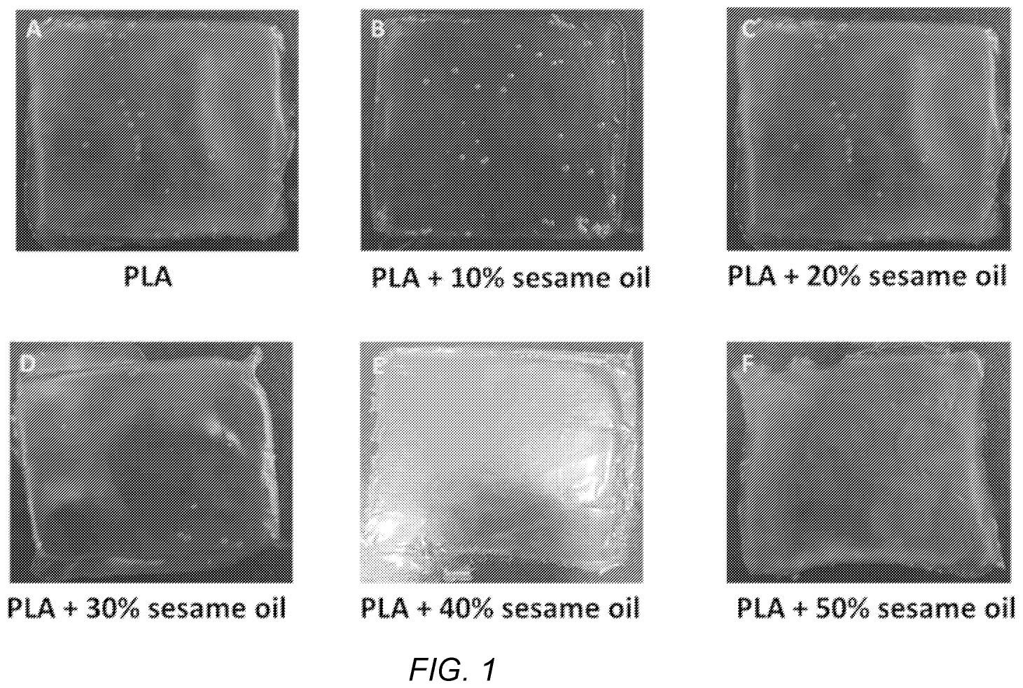

The PLA-oil films appeared dry and smooth by touch. As seen in FIG. 1, the transparency of the film decreases to near translucent with increasing amount of the sesame oil in the PLA. Further, the surface slight oiliness in the films is found only in films with 50% sesame oil. The absorbance of the sesame oil in the PLA matrix is unique and extraordinary. To the first time, we observed that the PLA has the tendency to absorb such a high amount of vegetable oil, in this case sesame oil. In the literature, the max amount of vegetable oil in PLA blend, used as plasticizer, that has been reported is up to 10% for the castor oil, soybean oil and cardanol oil, respectively. It should be understood that all this method of PLA-oil blend preparation involves melt blending which has limitation due to the phase inversion at low concentrations, leading to the loss of oils during blending with its increasing concentration. It was proposed that phase inversion is dependent on both thermodynamic and kinetic factors based on the observation that when identical blends were prepared with solvent-casting techniques, phase inversion was not observed, without loss in the oil content.

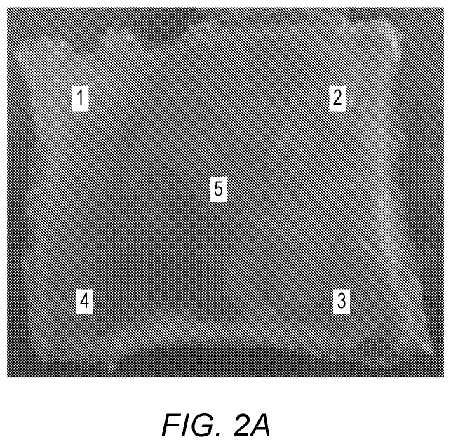

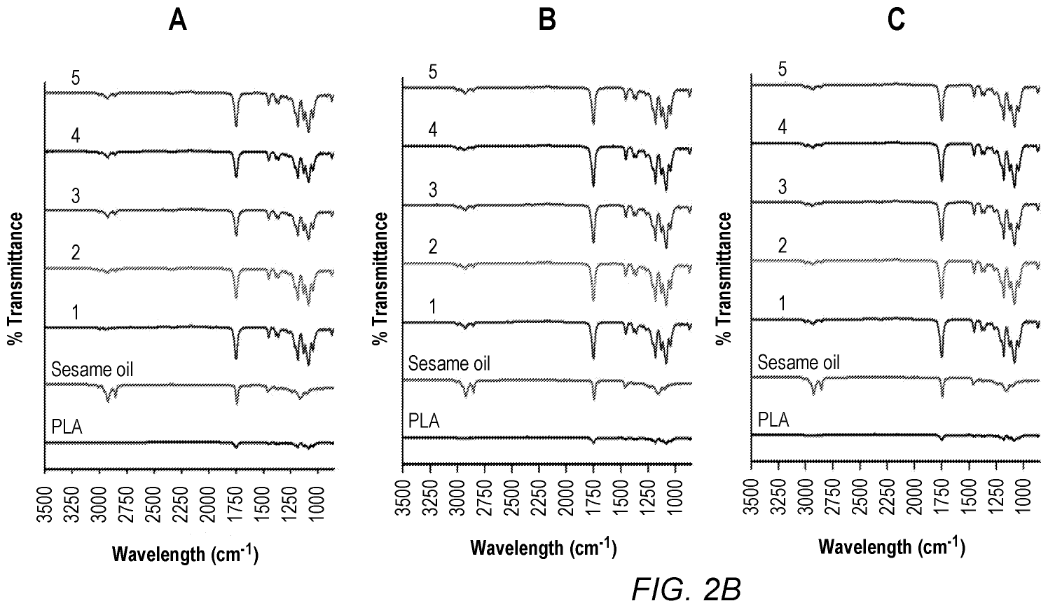

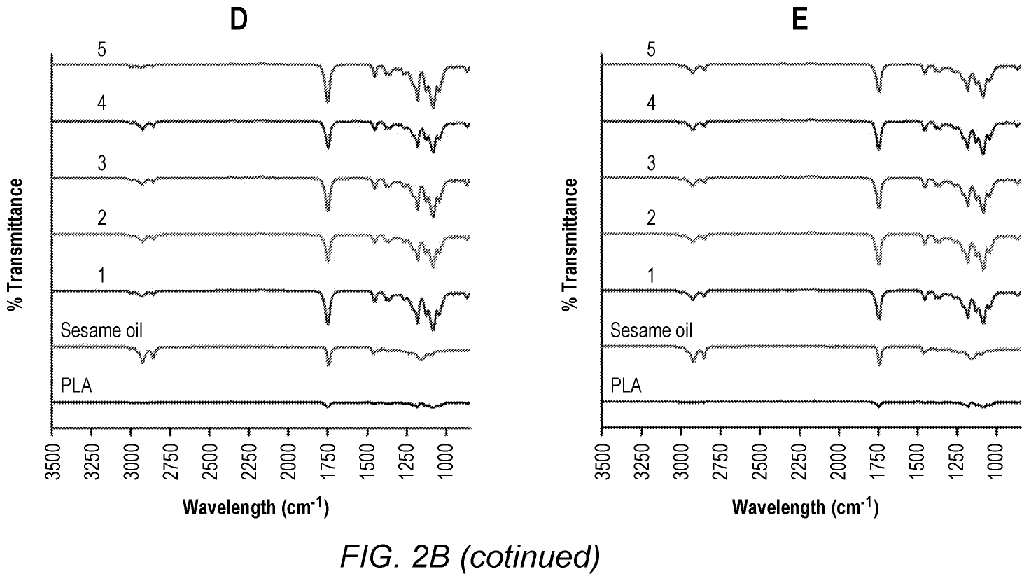

FTIR analysis: FTIR analysis of the films were performed in order to identify any change in the PLA composition. Further, homogeneous dispersion of the sesame oil can also be identified. The FTIR was performed from different regions of the films as shown in FIG. 2. Sesame oil alone has an absorption bands of triglycerides at 1744 cm⁻¹ (C═O of triglycerides or esters), Absorbance at 3008 cm⁻¹ associated with the stretching of cis double-bond (═CH). The sharp absorbance peaks from the asymmetric or symmetric stretching vibration of the aliphatic CH₂ groups is observed at around 2923 and 2854 cm⁻¹. Absorbance around 1462 and 1377 cm⁻¹ which result from bending vibrations of the CH₂ and CH₃ groups. The absorbance of 1098 cm⁻¹ is assigned with stretching vibration of the C—O ester group. On the other hand, PLA films showed the typical signal associated with the C—O stretch at ˜1087 cm⁻¹, C—O—C at ˜1183 cm⁻¹, O═C—O in ester groups at ˜1755 cm⁻¹, O═C—O stretch in ester groups at ˜1130 cm⁻¹ and CH₃ at ˜1458 cm. Because of the overlapping of the FTIR absorbance of the ester groups at 1755 cm “, the interaction between the oil and the polymers can be seen by examining the sharp absorbance peaks from the asymmetric or symmetric stretching vibration of the aliphatic CH₂ groups in sesame oil around ˜2923 and ˜2854 cm” 1. We see that the examination of various regions of the PLA sesame oil blended films, there was no shift observed in the ˜2923 and ˜2854 cm⁻¹. This indicated that there is no chemical interaction occurring between the sesame oil and the PLA. Also, presence of the ˜2923 and ˜2854 cm⁻¹ absorbance for the sesame oil in entire region of the films, indicate that the oil blends very homogeneously with the polymer too.

### Solid NMR Analysis

NMR experiments were performed on a Bruker Minispec 20 at 37° C. Measurements were performed on a Bruker Minispec 20 at 37° C. The longitudinal relaxation (T₁) was observed with a saturation recovery sequence. Free Induction Decay (FID), magic sandwich echo (MSE) and Hahn echo were recorded to characterize the transversal relaxation (T₂). The larger T₂ are more accurately measured with Hahn echo, the shorter T₂ with FID or MSE. The effect of sesame oil and other oils such as, lipiodol, poppyseed oil, olive oil, Miglyol, mineral oil, Witepsol H 32, and hydrogenated vegetable oils on PLA is given.

Transverse relaxation: The value of the 1H transverse relaxation time, i.e. the decay time of the NMR signal (FID), is a valuable parameter to evaluate the mobility of a system. The shorter the T₂ relaxation time, the less mobile the molecules are. Due to this unambiguous dependence between the NMR-relaxation time and the mobility of the system (correlation time Tc), conclusions about molecular dynamics can be drawn from T₂ measurements and cast into structural models. In crystalline systems, however, T₂ levels of at about 20 us which approximates the invers dipole-dipole coupling among 1H's. For slower molecular reorientations, the T₂ values remain constant and no dynamic information can be obtained anymore (so-called rigid limit). Long relaxation times are specific for mobile systems and may reach values of some milliseconds (ms) or even seconds in liquids. However, there is an upper limit of the T₂ values of about some ms in benchtop systems due to the inhomogeneity of the external magnetic field. This, the simple FID experiments have to be accompanied by echo experiments which overcome this limitation. Also, the first data points are often missing in FID experiments and make data processing more difficult. The Magic-sandwich echo (MSE) closes this gap. Thus, the combination of MSE, FID and Hahn enables the experimental determination a wide range of T₂ relaxation times. The different experiments FID (or MSE) and Hahn echo overlap on a reasonable time range and are always matched to each other in this overlap by the operator. For the sesame oil only the Hahn echo and FID was measured, since the missing early data points do not cause a problem in this mobile sample.

The T₂ decay of the neat PLA film contains the expected rigid signal for the crystalline part and a share of the amorphous part, while the additional slowly decaying component can be faithfully ascribed to the mobilized amorphous material due to the broadened glass transition which overlaps with the measurement temperature. The T₂ decays of the mixtures are basically superpositions of the individual components and three exponential decay functions with different coefficients β were used. The rigid phase is fitted to a gaussian shape, (β=2) while for the mobilized amorphous part, the β was set to 1. For the slowly relaxing component (oil), the exponent β is set variable, however, it appears to be close to 1 for all samples. There is no interphase component that could originate from a close interaction between oil and polymer. The T₂ values of the components in the mixtures are only slightly affected by the addition of sesame oil. This suggests that there are no major interactions between the oil and the polymer chains and the components are well phase separated (“physical mixture”). The mobilized polymer phase is found in the mixtures as well, albeit with alterations of the relative intensities and T₂ values, respectively.

In summary of this experiment, blending of vegetable oils to PLA and PCL was observed to be physical phenomenon. For PLA, no effect on both polymer and oil was observed. The mobility of the oil is strongly affected, while the polymer remains unaffected. Since T₁ relaxation behaviour is difficult to interpret, the data are interpreted only qualitatively. The T₁ relaxation times are only used to set up the T₂ relaxation time measurements. The T₂ components in NMR data fit to mass fractions.

DSC analysis: The DSC curves of the PCL-sesame oil blends were measured with a Mettler Toledo DSC 821e (Columbus, OH, USA). All samples were measured in the pan with lose cap (open system) and with pressed cap (closed system). The measurement program first heats up from −20° C. to 100° C. in 10 K/min, waits 1 min at 100° C., then back to −20° C. with 10K/min, waits for 2 min and again from −20 to 100° C. with 10K/min. The nitrogen flow was 20 ml/min. The DSC curves of polymer and oil mixtures exhibit minor changes in the melting peak with increasing oil content. The oil phase is clearly seen in the DSC, but does not change its melting behaviour. For PCL, the malting peak remain between 58 to 61° C. and the crystallinity between 48 to 45% while the delta H (J/g) decreases proportionally from 68 to 54 and 34 for the 0, 20 and 50% sesame oil in the blends. The crystallinities calculated from the DSC and the NMR measurements match well.

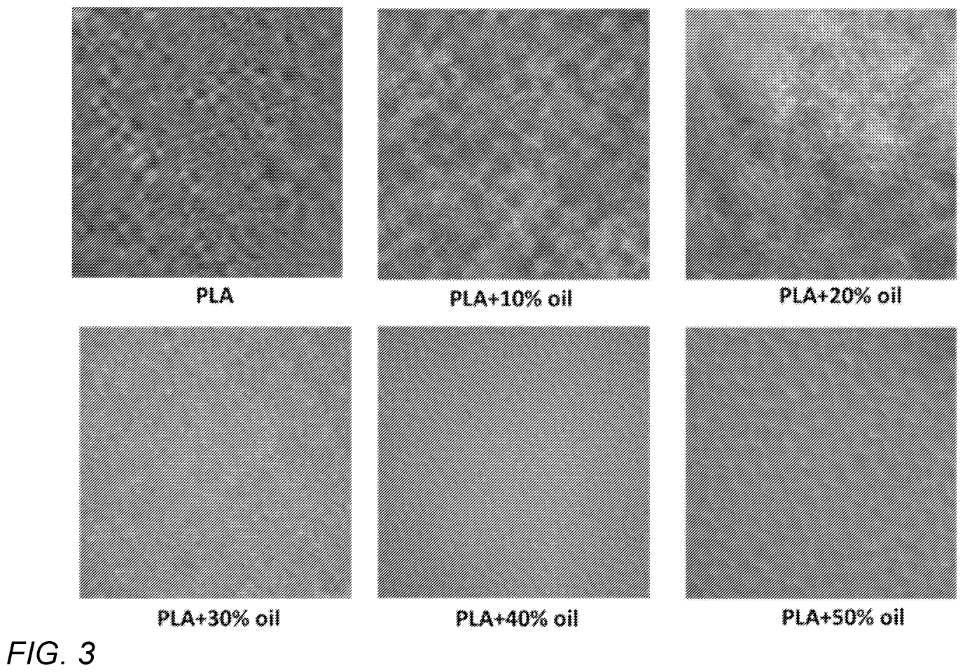

Confocal fluorescence analysis: Spinning disc confocal microscope (Nikon) was used for the fluorescence confocal analysis (x100) of the PLA-sesame oil films in the presence of Nile red was investigated. The amount of Nile red was as low as 0.01% in the films. Nile red was added to the pre-weighed sesame oil and PLA in the glass vials and chloroform was added to the blend. The sample was mixed over vortex for overnight at room temperature. A clear solution was obtained, and the mixture was kept drying at room temperature to evaporate the organic solvent in silicon molds covering with big beakers under closed hoods. This process helps in the removal of air bubbles that could formed due to condensation. The addition of the Nile red will show a color change under confocal microscopy imaging. We hypothesize that the Nile red is oil soluble, so, if the oil is heterogenetically distributed in the polymer there will be regions much colored and dense red in the films. Globules like structure appear in the presence of oil and more prominent with increasing oil concentration. It seems that the oil micro-drops are trapped on to the surface of the films while the organic solvents evaporate. As seen through FIG. 3 the size of the micro-drops increases with increasing amount of oil in the mixture. However, the distribution of the oil in the polymer films looks uniform even with increasing concentration. It should be noted that, the color of the Nile red intensified with the oil content. This we hypothesize is because of the accumulation of the Nile red in the oil bubbles at high concentration of the oil content in the PLA. At low concentration of the oil, the oil very well distributed into the polymer, hence, the microenvironment of the Nile red is less favorable in the polymer films with less oil content. With increasing oil content, the hydrophobicity in the films gradually increases, as well the formation of hydrophobic oil globules starts forming, suitable for the Nile red used as an in-situ marker provides high fluorescence in hydrophobic environment.

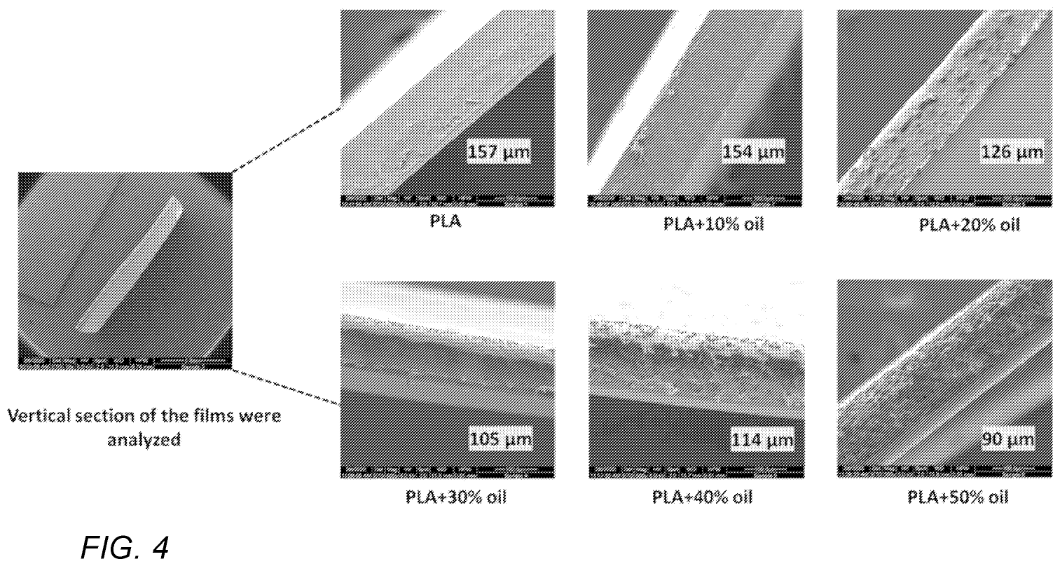

SEM analysis: Vertical cross-section of the PLA-sesame oil films was analyzed under scanning electron microscopy (SEM). The thickness of the films is dependent on the amount of the polymer available in the films. Thus, as shown in FIG. 4, a decrease in the film thickness occurred with increase in the amount of the oil content. Another thing that was notices is the oil-bulbs that appear when the films are formed. Supporting the fluorescence analysis, the size of the bulbs become more significant with increasing amount of the sesame oil in the films (FIG. 4). It has been reported in few of the literature that the bulb like formation is due to the large interfacial tension between oil and PLA. And this phenomenon seems to be independent of the preparation of the PLA-oil blend, as heat blending of the PLA-oil also show the formation of globules.

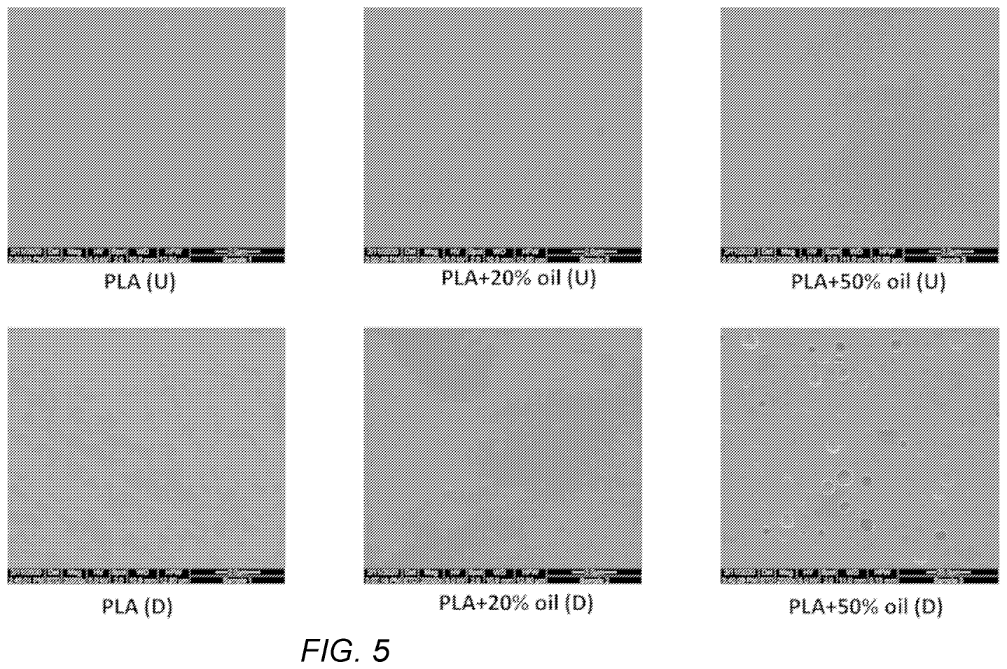

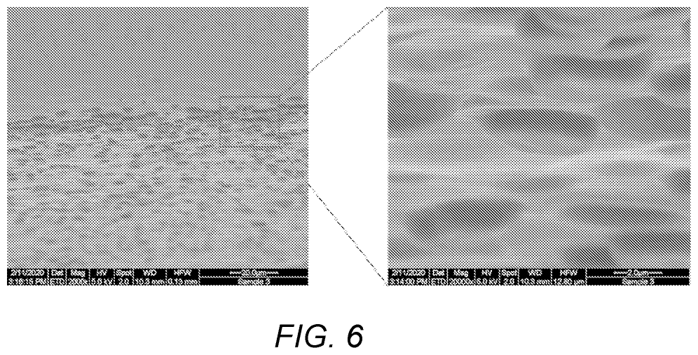

Further to get better understanding of the oil bulbs in the films we analyzed both the horizontal sides of the films which is shown in FIG. 5. We observe that the bulb appears only on one side of the horizontal surface. The film surface which is exposed to air while drying is smooth, while the other surface in contact with the silicon molds were having oil-bulbs. The bulbs are heavier due to the high density of the oil and settles down at the bottom while the organic liquid slowly evaporates. The size of the oil bulbs for 50% oil content in the film is observed to be 2-3 μm (FIG. 6).

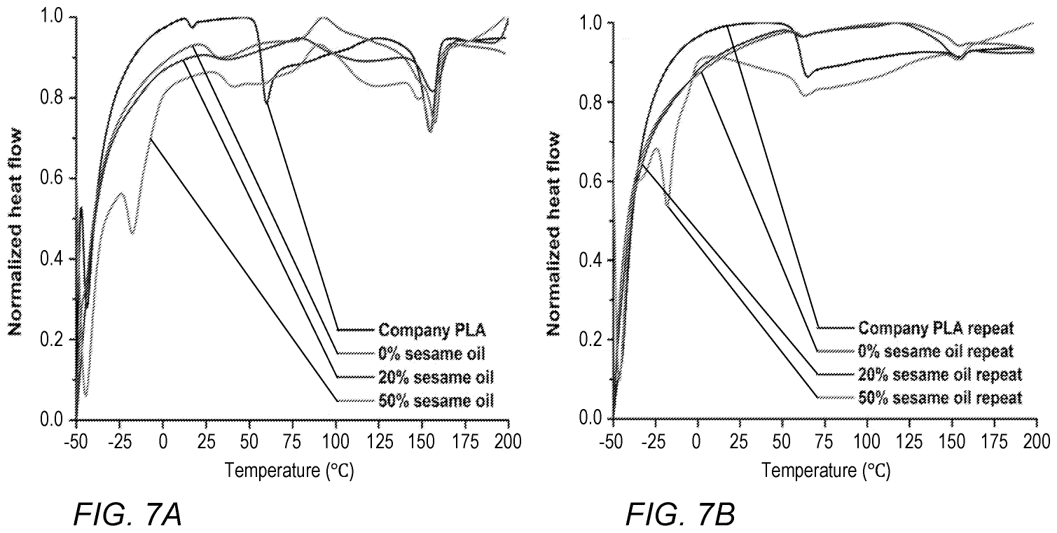

DSC analysis: Non-Isothermal Differential scanning calorimetry (DSC) was performed to investigate the changes in the polymer properties with the addition of the sesame oil. The rate of thermal scan was fixed to 10° C. per minute. After the thermal scan the samples were cooled by default setting of the machine. As represented in FIG. 7, during the first thermal scan, the melting temperature (Tₘ) of the PLA, and in the presence of 50% oil content appears at 155° C. However, when the second thermal scan was performed the crystallinity of the material is rapidly decreased as seen through decrease in the enthalpy of the system. This phenomenon is observed with the raw polymer (company polymer) too. Possibly, a non-isothermal cooling decreases the crystalline behavior of the PLA. Table 1 provides the thermal properties of the PLA-sesame oil films.

The degree of crystallinity was determined from the enthalpy of fusion (ΔH) which was calculated from the area under the endotherm.

TABLE 5
Thermodynamic data of the samples after first
thermal and second thermal (repeat) cycles.

| | | | | | | % Crystal- |
|---|---|---|---|---|---|---|
| | Tₘ | dH | % Crystal- | Tₘ (° C.) | dH (J/g) | linity |
| Sample | (° C.) | (J/g) | linity | (repeat) | (repeat) | (repeat) |
| Company PLA | 155.39 | −10.20 | 10.7 | 154.26 | −0.59 | 0.6 |
| 0% sesame oil- | 157.55 | −22.93 | 24.1 | 153.77 | −3.73 | 3.9 |
| PLA film | | | | | | |
| 20% sesame oil- | 157.22 | −16.00 | 16.8 | 155.34 | −9.99 | 10.5 |
| PLA film | | | | | | |
| 50% sesame oil- | 155.68 | −11.58 | 12.2 | 153.72 | −1.75 | 1.8 |
| PLA film | | | | | | |

Isothermal DSC analysis: The isothermal crystallization of PLA-sesame oil samples was recorded when the samples were heated from −50 to 200° C. and held at that temperature for 5 min to erase the thermal history, and then were cooled at a rate of 50° C./min to a certain isothermal crystallization temperature and were held until the crystallization finished. After the isothermal crystallization, the samples were heated at a rate of 10° C./min to 200° C. again for the thermal analysis. The results reveal that the isothermal treatment of the samples enhances the crystallinity of the PLA-sesame oil mixture (Table 6). The crystallinity of the polymer in the presence of oil is observed to increase after the isothermal treatment.

TABLE 6
Isothermal-thermodynamic data of the samples after
first thermal and second thermal (repeat) cycles.

| | | | | | | % Crystal- |
|---|---|---|---|---|---|---|
| | Tₘ | dH | % Crystal- | Tₘ (° C.) | dH (J/g) | linity |
| Sample | (° C.) | (J/g) | linity | (repeat) | (repeat) | (repeat) |
| Company PLA | 157.02 | −10.21 | 10.7 | 150.15 | −14.03 | 14.7 |
| 0% sesame oil- | 157.97 | −26.43 | 27.8 | 150.46 | −25.40 | 26.7 |
| PLA film | | | | | | |
| 20% sesame oil- | 155.80 | −15.81 | 16.6 | 149.78 | −20.75 | 21.8 |
| PLA film | | | | | | |
| 50% sesame oil- | 156.57 | −8.49 | 8.9 | 149.69 | −14.39 | 15.1 |
| PLA film | | | | | | |

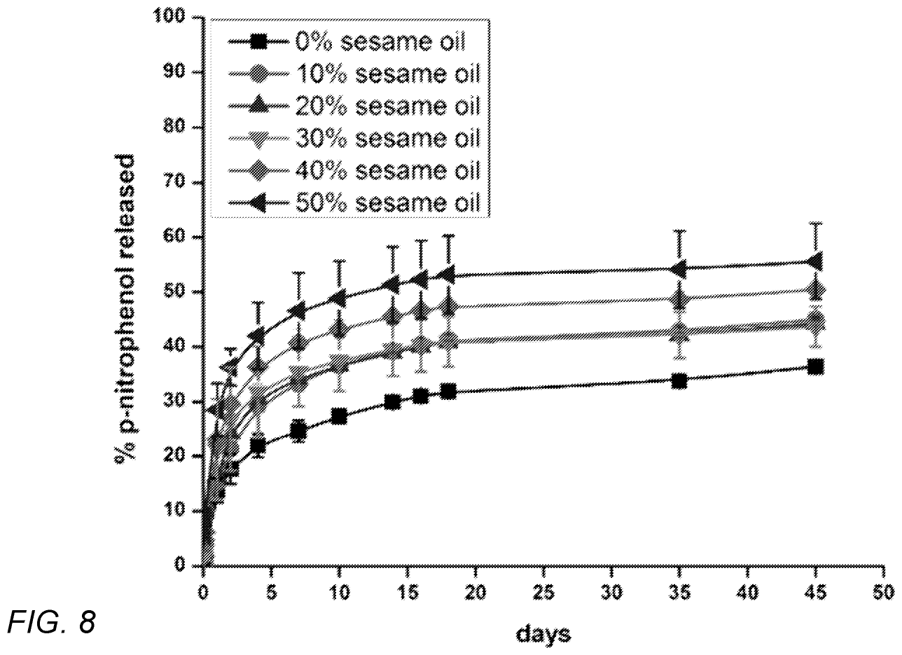

Release of oil soluble compounds from the PLA-sesame oil films: p-nitrophenol show good solubility in the sesame oil, hence, was considered for the release study. Additionally, the release of the p-nitrophenol will be dependent on the amount of oil coming into the release medium with time from the films. As shown in FIG. 8, the release of the p-nitrophenol is very slow, which means that the release of the sesame oil from the polymer matrix is relatively very low.

Elongation strength determination: In order to examine the changes in the mechanical behavior of the PLA films in the presence of sesame oil, we examined the resistivity of the films against elongation and the data is provided in Table 3. The increase in the elongation length of the film containing 20% sesame oil is due to the increase in the flexibility that is provided by the oil in the polymer matrix. Further, 50% films are relatively thinner, because of the decrease in the amount of the polymer, hence, they break easily compared to the polymer films with 20% sesame oil content.

TABLE 7
Elongation limits of the dog-bone shaped polymer films. The
data was collected by averaging results from 5 samples.

| | Average length | Average length when | |
|---|---|---|---|
| Sample Films | before elongation | elongation at break | |
| 0% sesame oil | 20 ± 2 mm | 25.58 ± 9.38 | mm |
| 20% sesame oil | 20 ± 2 mm | 31.4 ± 16.12 | mm |
| 50% sesame oil | 20 ± 2 mm | 24.67 ± 11.56 | mm |

Solid NMR analysis of PCL-sesame oil blends indicated the formation of a new composition that provides different spectra of the blends, depending on the oil content.

### Aqueous Degradation of the PLA-Sesame Oil Films:

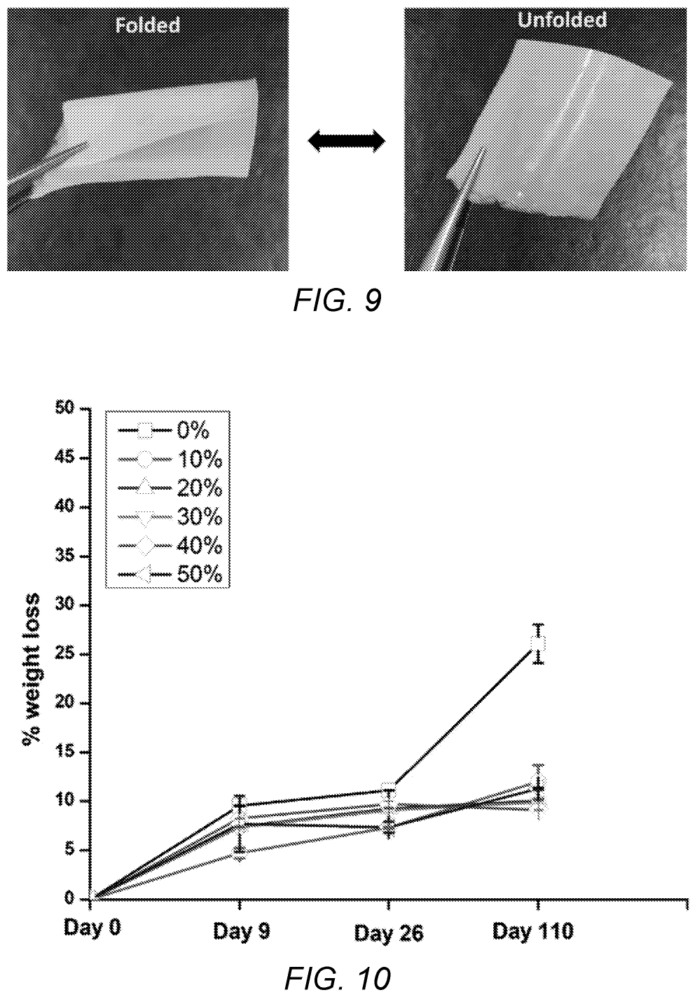

The appearance of the films hydrolytic degradation appears opaque white for all samples. Further, the PLA films containing 0, 10, 20, and 30% of the oil content became very brittle and instantly break by applying little pressure on it. On the other hand, PLA films with 40% and 50% sesame oil content remain foldable. As shown in FIG. 9 the films with 50% sesame oil content were folded without any crack.

% weight loss analysis: To understand the effect of aqueous hydrolysis of PLA in the presence of various amounts of sesame oil, we examined the weight loss in the PLA-sesame oil films at various time points. The films were placed in buffer medium (pH 7.2) of 10 mL and at 37° C. At each time point the films were taken out from the aqueous medium, soaked well over blotting paper and weighed. The wet films were then lyophilized, and the dry weight was taken again. FIG. 10 represents the weight loss in the PLA-sesame oil films. As seen through the FIG. 10, degradation of the blank PLA films is increased at day 110, compared to the films that contain the sesame oil. Probably the presence of the oil in the PLA films makes the films more hydrophobic and water repellant such that the polymer hydrolytic degradation is highly compromised. This slowing down of the hydrolytic degradation of the PLA is also seen in the literature. Et al observed that when blended with the soybean oil, the degradation behavior of the PLA-soybean was comparatively slow than the PLA itself [40]. Similarly, the presence of epoxidized soybean oil is observed to improves the PLA film stability against hydrolytic degradation.

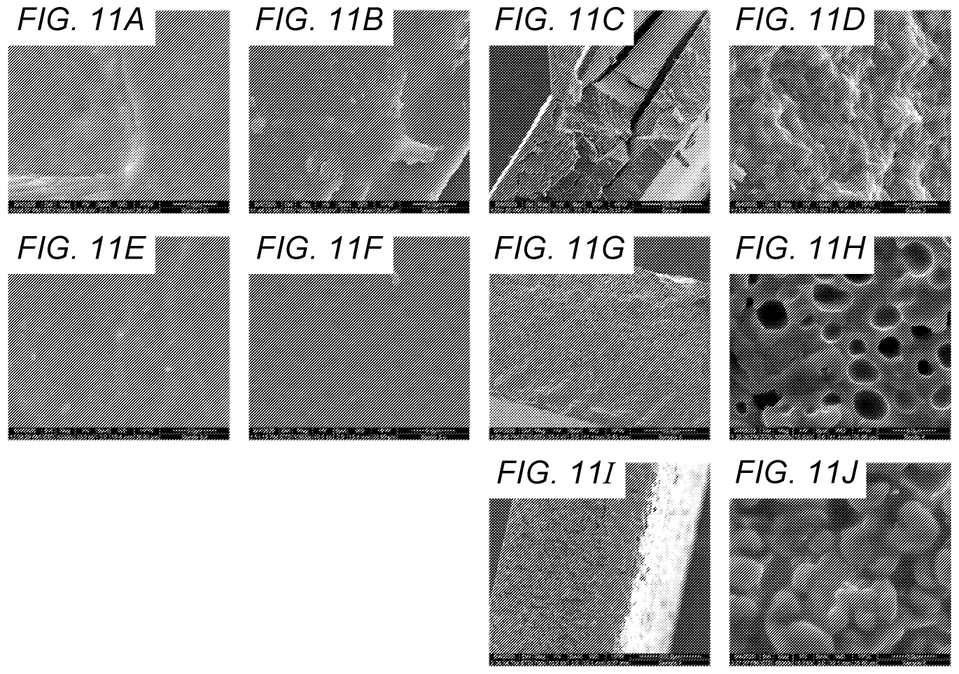

Characterization of the degraded films: SEM was performed to examine the morphological changes that occurred in the PLA-sesame oil films after 110 days of degradation. We examined films with 0%, 20% and 50% oil content. As seen through FIG. 11, The degradation of the PLA films occurs with the formation of pores allowing the water to disrupt the smoothness of the material. The deswelling on the oil bubbles in the PLA films with 20% sesame oil is observed, which upon cross-section examination reveal the formation of the micropores due to the degradation of the polymer, releasing the oil in the aqueous medium. This observation is similar to the results of the et al who proved that the globule are oil filled using cryomicrotoming process which removed the oil from the sample, leaving holes that are seen as dark circles surrounded by bright rings in SEM. Further, the oil globules remain intact with the PLA films with 50% sesame oil content. Herein, we see the existence of the oil globules as seen in FIG. 7. This is the reason that the PLA films with 40 and 50% oil content remained flexible enough to fold. Additionally, the oil globules made the surface highly hydrophobic and water repellant, that compromised the rate of hydrolytic degradation.

### Example 3: Preparation of Polymer-Oil Blends Microparticles Loaded with Escitalopram

Cipralex is a brand name for the drug escitalopram (ESC), that is antidepressant drug that belong to the group of selective serotonin reuptake inhibitors (SSRIs). Developing ESC controlled release drug delivery system is important in order to decrease administration frequency, to achieve sustained release of the drug, and by that to improve the patient compliance. The polymeric microparticles is an example of the drug delivery system that can be developed, the particles must be prepared of biocompatible and biodegradable polymers, for example poly(lactic acid) (PLA) and poly(lactic-co-glycolic acid) (PLGA). The release rate of the drug can be tuned by the type and the ratio of the oil used in the preparation of the particles. In addition, the presence of the oil can improve the drug encapsulation by improving the interaction with hydrophobic drugs. The aim of this study was to develop and characterize polymer-oil microparticles loaded with ESC, and to determine the effect of the oil on the drug loading and release profile of the obtained microparticles.

### Materials

Poly lactic acid (PLA) (100 kDa) from Nature Work LLC; PLGA 75:25 (100 kDa) from Carbion Purac; Escitalopram free base (Cipralex) was available in the lab; Dichloromethane (DCM) purchased from Bio-lab; Polyvinyl alcohol PVA, MCT oil, castor oil, and sesame oil was available in the lab

### Methods

Preparing the microparticles: The microparticles prepared by oil in water (o/w) emulsion solvent evaporation method. For this, 20 mg of the polymer, 20 mg of the oil and 10 mg of the drug dissolved in 1 ml DCM. The organic solution was added dropwise into 20 ml of 0.2% PVA aqueous solution. The mixture stirred at 600 rpm for overnight for evaporation the solvents. The particles collected by decantation or centrifuging the mixture at 5000 rpm (unless specified) for 15 min, the supernatant removed carefully, and the sediment particles washed one time with 10 ml DDW then centrifuged again at 5000 rpm for 15 min, the supernatant removed, and the collected particles were lyophilized to obtain dry material.

ESC standard curve: ESC 1 mg/ml concentration prepared in 2 ml ethanol, then 8 ml of PBS pH 7.2 added to achieve ESC concentration of 200 μg/ml. From the stock solution, multiple concentration prepared in the range of 1-20 μg/ml in PBS. The absorption of the prepared concentration measured at 238 nm using UV spectrophotometer.

Determining the drug loading: Drug loading was determined by centrifuging 1 ml of the microparticle dispersion at 13500 rpm for 15 min, then the absorption of the supernatant was measured at 238 nm.

Scanning electron microscopy (SEM): Morphology of the prepared microoparticles was observed under QUANTA 200 scanning electron microscopy (SEM). The sample was placed on double sided conductive carbon adhesive tape fixed on a metal stub. The metal stubs containing samples were vacuum coated with a thin layer of gold, using ion sputter with Au target assembly. The analysis was operated at a voltage of 10.00 kV.

In vitro Release: The in vitro release study conducted by transferring 10 mg of the microparticles dry material into 2 ml Eppendorf vial with 2 ml of phosphate buffer (10 mM and pH 7.2) at 37° C. and shaking of 75 rpm. The release medium was replaced periodically with fresh buffer after centrifuging at 13500 rpm for 10 min and ESC content in the solution was determined by measuring the absorption at 238 nm.

### Results and Discussion

For the preparation of the microparticles, oil in water emulsion evaporation method was used. The polymer, oil and the drug were dissolved in 1 ml DCM. The components of all formulations are listed in Table 8. The ESC loading determination show that the PLA: MCT: ESC 20:20:10 by weight in mg (in 1 mL DCM) has the highest drug loading with 16%, the other oils show loading between 12-14% for the PLA particles and 11% for the PLGA particles, as seen in Table 8. In addition, the oils increased the loading of the particles compared to the pure polymer particles.

TABLE 8
The composition and the drug loading
of the prepared microparticles

| Polymer | | Polymer:Oil:ESC | Drug |
|---|---|---|---|
| type | Oil type | [mg:mg:mg] | loading* |
| PLA | — | 20:0:5 | 10% |
| PLA | MCT | 20:20:10 | 16% |
| PLA | MCT | 20:10:10 | 13% |
| PLA | Sesame | 20:20:10 | 12% |
| PLA | Castor | 20:20:10 | 13% |
| PLGA | — | 20:0:5 | 5% |
| PLGA (75:25) | MCT | 20:20:10 | 11% |

*the actual ESC content in the isolated microspheres was determined by UV. Escitalopram was extracted for the microspheres and determined by UV. The oil content in the polymers was 50% w/w and the original drug loading was 20% w/w

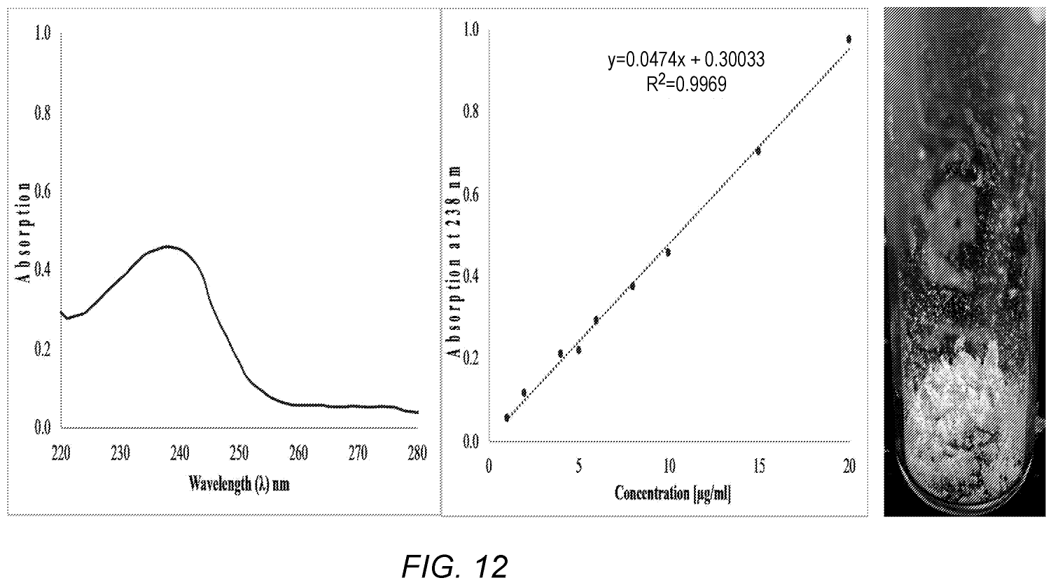

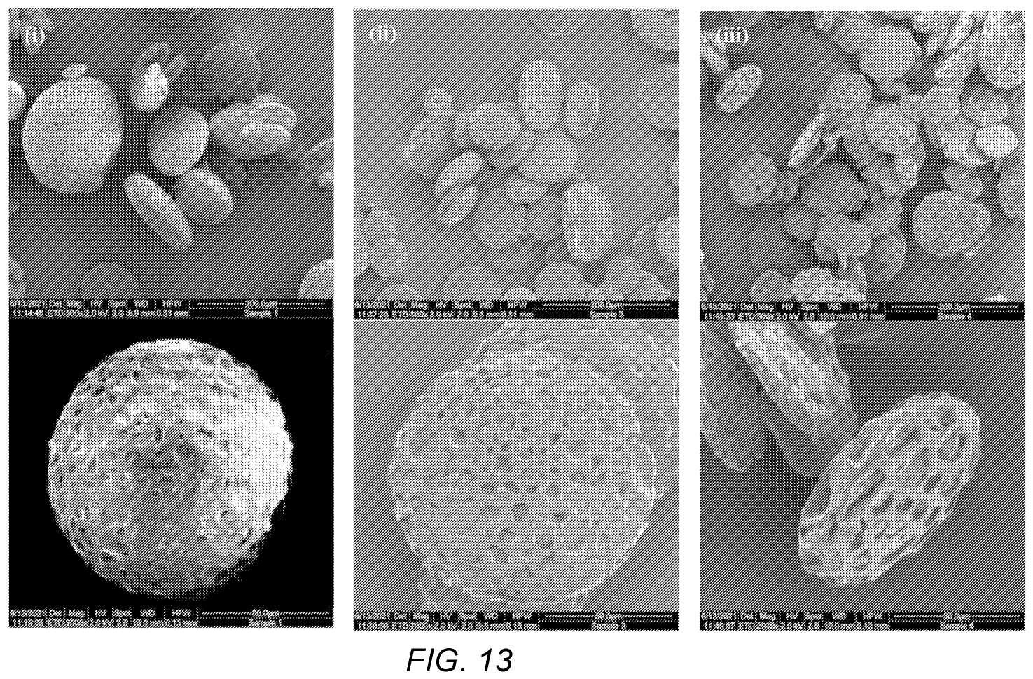

The standard curve of ESC was done in PBS pH 7.2 and the absorption measured using UV spectroscopy at 238 nm, the curve show linearity with concentration range between 1-20 μg/ml, as provide in FIG. 12-ii. The prepared particles were obtained as white powder as seen in FIG. 12-iii, the particles prepared with sesame oil were a bit oily, the particles prepared with MCT were less oily, and the castor particles were the least oily particles. The dispersity of the obtained particles in aqueous solution varied due to the difference in polymer type as seen in FIG. 13, the PLGA particles show better dispersity, this could be due to the fact that PLGA is more hydrophilic than PLA. The oil used in the preparation also affect the dispersity, particles prepared with castor oil show the better dispersity.

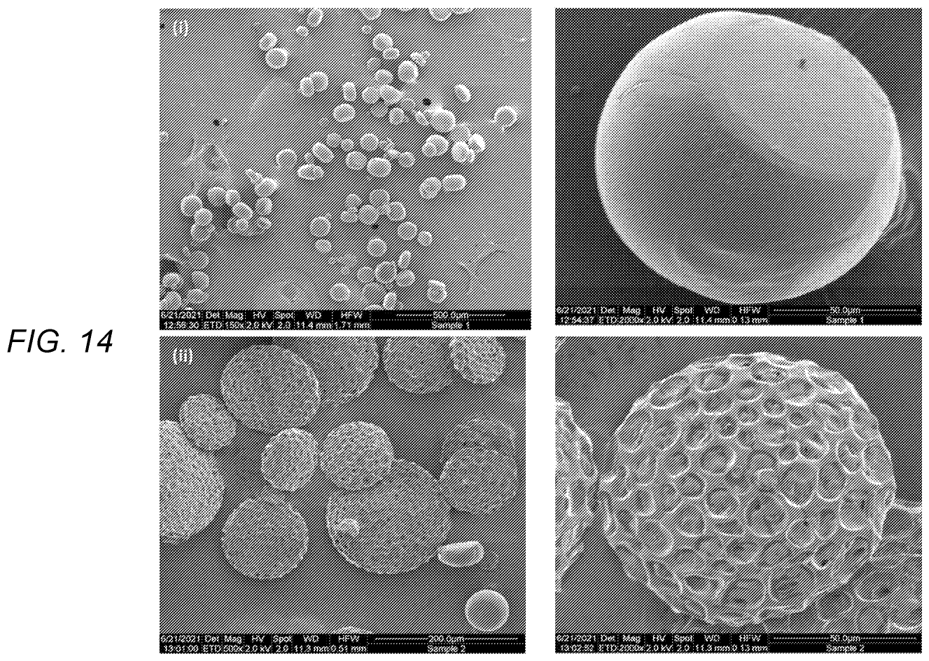

The morphological properties of the obtained particles are important factor that influence the drug loading and release, the morphological properties of the particles were determined using SEM as provide in FIG. 13. The SEM images show that the particles are round in shape, and the size was less than 50 μm. The SEM image in FIG. 13 show that particles prepared using PLA polymer have biconcave disk like shape and not spherical. The particles prepared with MCT show the less condensed shape as seen in FIG. 13-i. However, the particles prepared sesame oil are the most condensed particles and they look very thin as seen in FIG. 14-ii. The observed shape could be as a result of the presence of oil inside the particles that affect their mechanical strength and lead them to collapse.

For further understanding of the effect of the preparation parameter on the obtained particle shape, blank PLA particles prepared with polymer to oil ratio of 1:0 and 1:1, the difference in the preparation was in collecting the microparticles, for these two formulations the particles were collected by decantation and not centrifugation in order to reduce the force applied on the particles. The PLA particles with 1:0 polymer to oil ratio have almost a spherical shape as seen in FIG. 14-i. However, PLA particles with 1:1 polymer: oil ratio presented in FIG. 14-ii, have the biconcave shape as similar to the particles prepared previously and presented in FIG. 13, this mean that the observed shape is a result of the presence of oil.

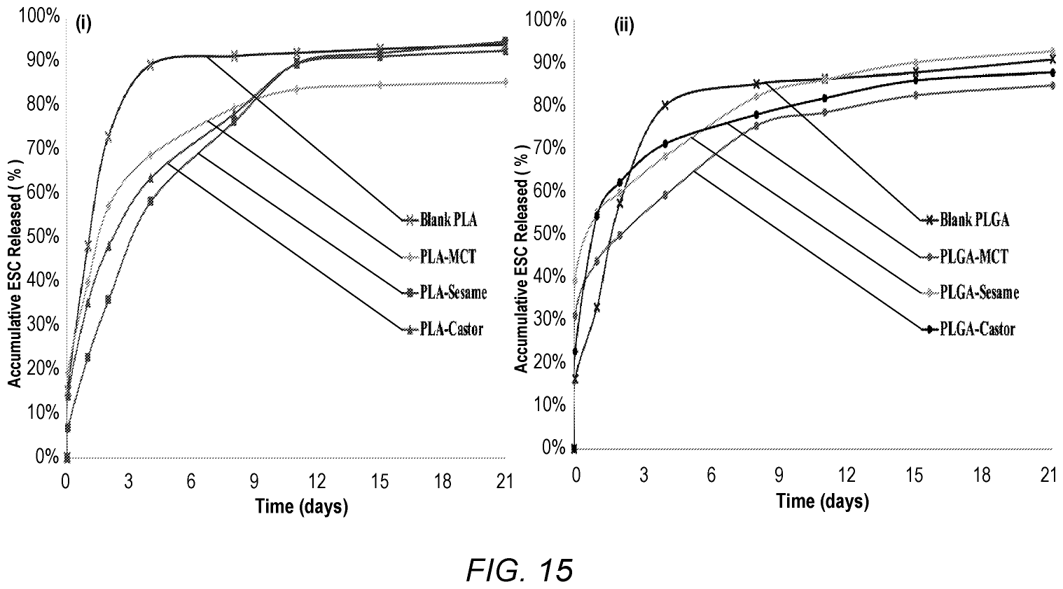

In the in vitro release study, 10 mM PBS pH 7.2 was used as the release medium for the ESC from the developed microparticles. The release result show that over 80% of the drug was released over a period of 20 days in all the formulation as seen in FIG. 15. In addition, the loading after the release experiment was measured at the day 21^{st} by placing the release sample on strong vortex for 48 h, after that the samples were sonicated for 15 min. The release vials centrifuged at 13500 rpm for 15 min and the supernatant absorption measured at 238 nm. The release rate of ESC from PLA and PLGA particles initiated with a burst release that varied between the samples as a result of the different oil. As presented in FIG. 15, the PLA-MCT released around 20% of the drug in the first day, and PLA sesame oil only 10% of the drug in the first day. The release study was repeated after preparing new the formulation and collecting the particles by decantation.

The results show that oil-polymer microparticles could be used as an alternative to pure polymer microparticles, the oil-polymer particles improved the drug loading compared to the pure polymer particles. In addition, from the different oil used in the preparations, MCT achieve the highest ESC loading. The microparticles released over 80% of the loaded ESC over a period of 21 days. The microparticles collected by decantation or by centrifugation. Different oil could be used to determine the appropriate oil that achieve the highest loading and the best release profile for both polymers.

Preparing PLA-Oil Blends Microparticles Loaded with Escitalopram

In the previous part of the study escitalopram loaded microparticles made of PLA and PLGA with oil was prepared and characterized, the oil ratio in the particles was 40% (w/w). In this part of the study, PLA microparticles were prepared with different oily materials with higher melting point, and sesame oil-microparticles that was prepared above but with oil content between 10-40% w/w. The obtained particles were characterized by SEM, and a release was determined.

### Materials

Poly lactic acid (PLA) (100 kDa) from Nature Work LLC; Escitalopram free base (Carpalia) was available in the lab; Dichloromethane (DCM) purchased from Bio-lab; Polyvinyl alcohol PVA, sesame oil (liquid), stearic acid (melting point 69° C.), cetyl alcohol (melting point 50° C.), coconut oil (melting point 240 C), and hydrogenated vegetable oil, VGB6 (melting point-68-74° C.) was available in the lab

### Methods

Preparation of microparticles: The microparticles were prepared by oil in water (o/w) emulsion solvent evaporation method. For this 20 mg of the polymer, 20 mg of the oil and 10 mg of the drug dissolved in 1 ml DCM. The organic solution was added dropwise into 20 ml of 0.2% PVA aqueous solution. The mixture stirred at 600 rpm for overnight for evaporation the solvents. The particles collected by decantation in ice for 4-6 hours, the supernatant was removed carefully, and the sediment particles washed one time with 10 ml DDW then collected again by decantation, the collected particles were lyophilized to obtain a dry powder.

ESC standard curve: ESC 1 mg/ml concentration prepared in 2 ml ethanol, then 8 ml of PBS pH 7.2 added to achieve ESC concentration of 200 μg/ml. From the stock solution, multiple concentration prepared in the range of 1-20 μg/ml in PBS. The absorption of the prepared concentration measured at 238 nm using UV spectrophotometer.

Determining the drug loading: Drug loading was determined by centrifuging 1 ml of the microparticle dispersion at 13500 rpm for 15 min, then the absorption of the supernatant was measured at 238 nm.

Scanning electron microscopy (SEM): Morphology of the prepared microparticles was observed under QUANTA 200 scanning electron microscopy (SEM). The sample was placed on double sided conductive carbon adhesive tape fixed on a metal stub. The metal stubs containing samples were vacuum coated with a thin layer of gold, using ion sputter with Au target assembly. The analysis was operated at a voltage of 10.00 kV.

In vitro Release: The in vitro release study conducted by transferring 10 mg of the microparticles dry material into 2 ml Eppendorf vial with 2 ml of phosphate buffer (10 mM and pH 7.2) at 37° C. and shaking of 75 rpm. The release medium was replaced periodically with fresh buffer after centrifuging at 11000 rpm for 15 min and ESC content in the solution was determined by measuring the absorption at 238 nm.

### Results and Discussion

For the preparation of the microparticles, oil in water emulsion evaporation method was used. The components of all formulations are listed in table 1. The ESC loading determination show that the PLA microparticles with 10% sesame oil w/w achieved the highest loading of 14% over the rest of the prepared microparticles. In addition, the reduction in the oil amount used in the preparation increased the drug loading as seen in Table 1 for the sesame oil sample. The samples prepared with VGB6 showed the lowest loading of 3%.

TABLE 9
The composition and the drug loading
of the prepared microparticles

| Polymer | | Polymer:Oil:ESC | Oil content | Drug |
|---|---|---|---|---|
| type | Oil type | [mg:mg:mg] | (w/w) | loading* |
| PLA | — | 20:0:5 | 0% | 10% |
| PLA | Coconut | 20:20:10 | 40% | 7% |
| PLA | Cetyl alcohol | 20:20:10 | 40% | 6% |
| PLA | Stearic acid | 20:20:10 | 40% | 13% |
| PLA | VGB 6 | 20:20:10 | 40% | 3% |
| PLA | Sesame oil | 20:20:10 | 40% | 11% |
| PLA | Sesame oil | 20:15:10 | 30% | 11% |
| PLA | Sesame oil | 20:7.5:10 | 20% | 13% |
| PLA | Sesame oil | 40:7.5:20 | 10% | 14% |

*the actual ESC content in the isolated microspheres was determined by UV. Escitalopram was extracted for the microspheres and determined by UV. The oil content in the polymers was 50% w/w and the original drug loading was 20% w/w

The prepared particles were obtained as white powder. The particles prepared with stearic acid obtained as big particles in the mm scale. The particles prepared with VGB6 showed also big particles that break easily. The dispersity of the obtained particles in aqueous solution varied due to the difference in the oil type. The particles made with coconut oil showed the best dispersity, followed by the cetyl alcohol particles. The particles made with stearic acid and VGB 6 were not dispersed well.

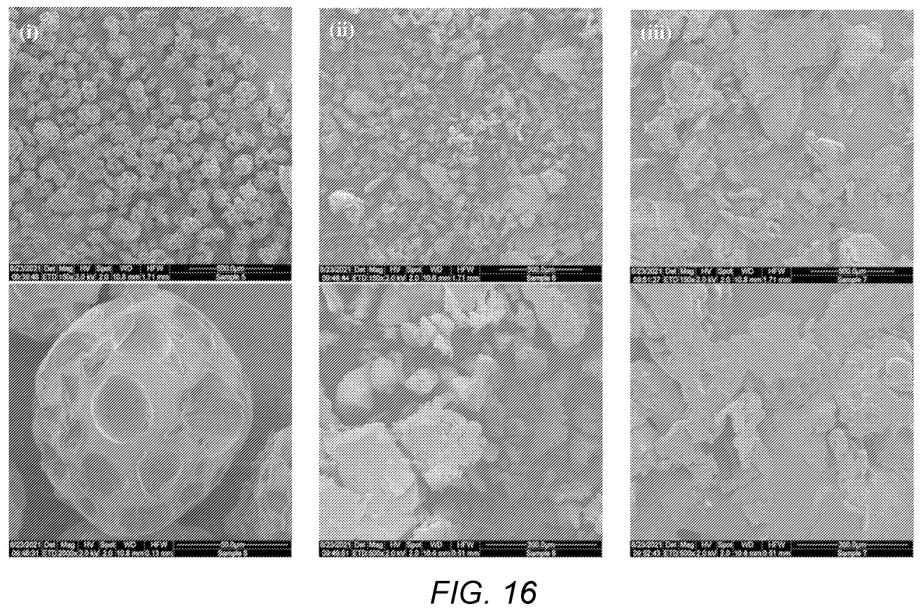

The morphological properties of the obtained particles were determined using SEM. The SEM images of the particles made with coconut oil presented in FIG. 16-I, have biconcave-like shape, similar to the particles made with MCT, castor and sesame oil. The particles made with cetyl alcohol and stearic acid are big chunks as seen into FIG. 16-ii & iii.

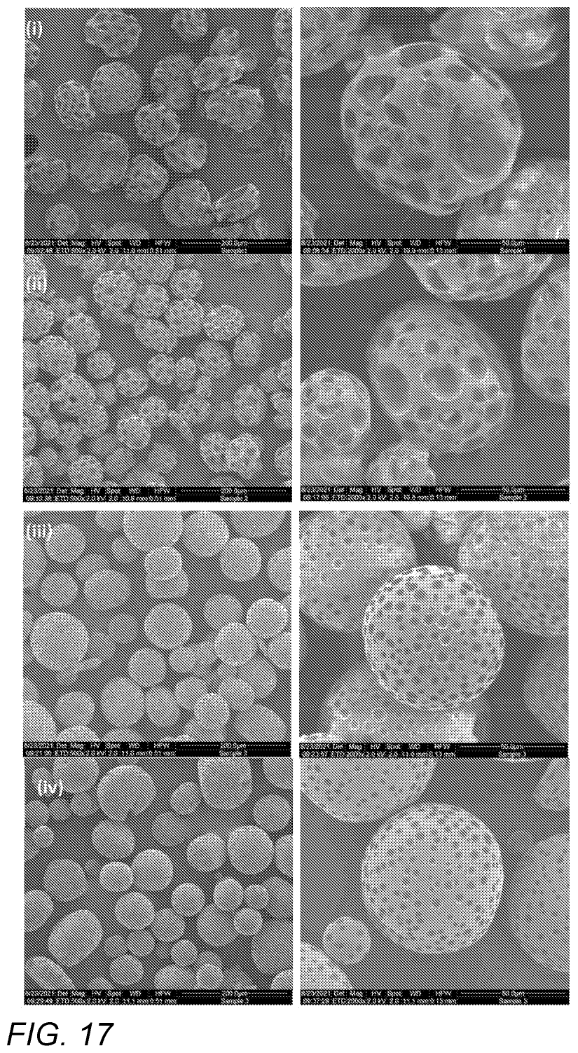

The morphological properties of the particles prepared with different sesame oil ratio was visualized by SEM (FIG. 17). All particles are similar in shape and size. The lower the oil content the smaller are the holes on the particle surface.

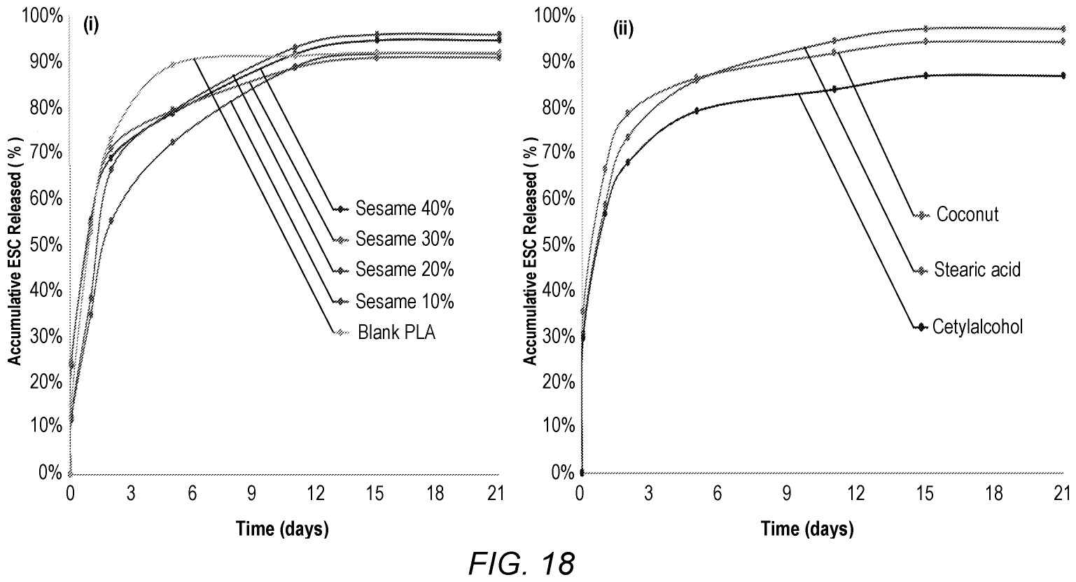

In the in vitro release study, 10 mM PBS pH 7.2 was used as the release medium for the ESC from the developed microparticles. The release result show that over 90% of the drug was released over a period of 20 days in all formulations as seen in FIG. 18. In addition, the loading after the release experiment was measured at the day 21^{st} by placing the release sample on strong vortex for 48 h, after that the samples were sonicated for 15 min. The release vials centrifuged at 13500 rpm for 15 min and the supernatant absorption measured at 238 nm. The release rate of ESC from PLA-sesame particles varied between the sample as a result of the different in the sesame concentration, the particles with 10% sesame showed the slowest release profile among the other higher sesame concentration. The sample prepared with the other type of oil showed faster release that initiated with burst release.

The results of this study showed that high melting lipids are less suitable for preparing microspheres for drug delivery. Different oil content provided similar particle size and shape with some differences in drug release rate.

### Example 4: Preparation and Characterization of Oil Blends Polymeric Films

### INTRODUCTION

The aim of this study was to prepare polymer-oil blends film using different types of oil in concentration of 10%, 30% and 50% (w/w), and to characterize the physical and mechanical properties of the prepared films. In addition, different analysis method will be used to analyze the prepared films, for instance, FTIR, NMR, DSC, SEM, and other methods.

### Materials

Polymers: Poly lactic acid (PLA) (100 kDa) from Nature Work LLC; PLGA 75:25 (100 kDa) from Carbion Purac, Polycaprolactone (PCL) (80 kDa)

Oils: sesame oil, castor oil and VGB 4 available in the lab, witepsol E85 and witepsol H32 from IOI oleochemical, coconut from across organic, stearic acid from sigma.

Solvents: Chloroform (CHF) from sigma

### Methods

Preparing the films: The films were prepared by dissolving polymer and oil in the appropriate amount of chloroform to achieve the concentration of 100 mg/mL. The sample placed on vortex for overnight to fully dissolve the materials. Then 1 ml of the solution was injected into 2x2 cm² silicon mold covered with beaker in the hood to control the evaporation and reduce the amount of air bubbles in the films. The films were kept to dry at room temperature overnight to fully evaporate the organic solution.

Physical examination: The stability and the compatibility of the prepared films were examined by touch and with visual appearance. The films showed dry surface, flexible and transparent.

Characterization with FTIR: FTIR of the prepared films were performed to identify any change in the polymer composition. In addition, the homogeneity of the oil dispersion in the films can be identified by performing the FTIR on different region of the prepared films.

Degradation: The stability of the prepared blends films in aqueous solution determined by placing the films into 20 ml glass vial with 20 ml of DDW, the vial placed in the oven at 37° C. for 4 weeks. the films were checked weekly by visualization to determine any change. FTIR and SEM were performed.

### Results and Discussion

The properties of the films and from polymer-oil are given in Table 10 to 14. Most films are partially transparent. The films are surface dry without surface oil feel. FTIR data represented both the oils and the polymers.

TABLE 10
The composition and physical properties
of the prepared PLA-oil blend films

| | Polymer | oil | Oil | visual | brittleness |
|---|---|---|---|---|---|
| Oil | [mg] | [mg] | ratio | appearance | after folding |
| Sesame | 120 | 120 | 50% | partially | No |
| oil | | | | transparent | |
| | 120 | 52 | 30% | partially | No |
| | | | | transparent | |
| | 120 | 14 | 10% | transparent | No |
| Witepsol | 120 | 120 | 50% | partially | yes |
| H32 | | | | transparent | |
| | 120 | 52 | 30% | almost | No |
| | | | | transparent | |
| | 120 | 14 | 10% | transparent | No |
| Witepsol | 120 | 120 | 50% | partially | Yes |
| E85 | | | | transparent | |
| | 120 | 52 | 30% | partially | No |
| | | | | transparent | |
| | 120 | 14 | 10% | transparent | No |
| | 120 | 120 | 50% | Non-transparent | Yes |
| Stearic | 120 | 52 | 30% | Non-transparent | Yes |
| acid | 120 | 14 | 10% | Non-transparent | No |
| Castor | 120 | 120 | 50% | partially | Not |
| oil | | | | transparent | |
| | 120 | 52 | 30% | partially | not |
| | | | | transparent | |
| | 120 | 14 | 10% | partially | not |
| | | | | transparent | |
| Coconut | 120 | 120 | 50% | partially | Yes |
| oil | | | | transparent | |
| | 120 | 52 | 30% | transparent | No |
| | 120 | 14 | 10% | transparent | No |
| VGB 4 | 120 | 120 | 50% | not transparent | Yes |
| | 120 | 52 | 30% | not transparent | Yes |
| | 120 | 14 | 10% | not transparent | Yes |

Polycaprolactone (PCL) films. The properties of the various films are given in Table 11. All films are not transparent but white. Most are not brittle and do not have a surface-feel of oil.

TABLE 11
The composition and physical properties
of the prepared PCL-oil blend films

| | | | | | oily | brittleness |
|---|---|---|---|---|---|---|
| | Polymer | oil | oil | visual | surface | after |
| Oil | [mg] | [mg] | ratio | appearance | (by touch) | folding |
| Sesame | 130 | 130 | 50% | white film | Yes | Yes |
| oil | 130 | 55 | 30% | white film | Yes | No |
| | 130 | 15 | 10% | white film | No | No |
| Witepsol | 130 | 130 | 50% | white film | Yes | Yes |
| H32 | 130 | 55 | 30% | white film | No | No |
| | 130 | 15 | 10% | white film | No | No |
| Witepsol | 130 | 130 | 50% | white film | No | No |
| E85 | 130 | 55 | 30% | white film | No | No |
| | 130 | 15 | 10% | white film | No | Yes |
| Stearic | 130 | 130 | 50% | white film | Yes | Yes |
| acid | 130 | 55 | 30% | white film | Yes | Yes |
| | 130 | 15 | 10% | white film | No | No |
| Castor | 130 | 130 | 50% | partially | Yes | No |
| oil | | | | transparent | | |
| | 130 | 55 | 30% | partially | No | No |
| | | | | transparent | | |
| | 130 | 15 | 10% | partially | No | No |
| | | | | transparent | | |
| Coconut | 130 | 130 | 50% | white film | Yes | No |
| oil | 130 | 55 | 30% | white film | No | No |
| | 130 | 15 | 10% | white film | No | No |
| VGB 4 | 130 | 130 | 50% | white film | Yes | Yes |
| | 130 | 55 | 30% | white film | Yes | No |
| | 130 | 15 | 10% | white film | Yes | No |

PLGA (75/25) films The properties of the various films are given in Tables 4. Most films are fully transparent. Most are not brittle and do not have a surface-feel of oil.

TABLE 12
The composition and physical properties of
the prepared PLGA(75/25)-oil blend films

| | | | | | | oily | brittleness |
|---|---|---|---|---|---|---|---|
| | | Polymer | oil | oil | visual | surface | after |
| Polymer | oil | [mg] | [mg] | ratio | appearance | (by touch) | folding |
| PLGA | Sesame | 120 | 120 | 50% | transparent | Yes | No |
| 75:25 | oil | 120 | 52 | 30% | transparent | No | No |
| | | 120 | 14 | 10% | transparent | No | No |
| | Witepsol | 120 | 120 | 50% | transparent | Yes | No |
| | H32 | 120 | 52 | 30% | transparent | No | No |
| | | 120 | 14 | 10% | transparent | No | No |
| | Witepsol | 120 | 120 | 50% | Not transparent | Yes | No |
| | E85 | 120 | 52 | 30% | Almost | | |
| | | | | | transparent | | |
| | | 120 | 14 | 10% | Almost | No | No |
| | | | | | transparent | | |
| | Stearic | 120 | 120 | 50% | white film | Yes | Yes |
| | acid | 120 | 52 | 30% | white film | Yes | Yes |
| | | 120 | 14 | 10% | white film | Yes | No |
| | Castor | 120 | 120 | 50% | transparent | Yes | No |
| | oil | 120 | 52 | 30% | transparent | Yes | No |
| | | 120 | 14 | 10% | transparent | No | No |
| | Coconut | 120 | 120 | 50% | transparent | Yes | No |
| | oil | 120 | 52 | 30% | transparent | Yes | No |
| | | 120 | 14 | 10% | transparent | No | No |
| | VGB 4 | 120 | 120 | 50% | white film | Yes | Yes |
| | | 120 | 52 | 30% | white film | Yes | No |
| | | 120 | 14 | 10% | white film | No | No |

### Example 5: Preparing Polymeric-Oil Blends by the Heat Blending Method

The blending method was used to improve the polymer physical and mechanical properties without affecting the chemical properties of the polymers. Heat blending was used to prepare polymer-oil blends.

### Materials

Polymers: Poly lactic acid (PLA) (100 kDa) from Nature Work LLC; Polycaprolactone (PCL) (80 kDa)

Oils: sesame oil, castor oil, and MCT oil available in the lab, witepsol E85 and witepsol H32 from IOI oleochemical, coconut from across organic, stearic acid from sigma.

### Methods

Preparing the blends of PCL: 500 mg of the polymer placed in 20 ml glass vial and the placed into oil bath at 100-120° C. until the polymer melted. 500 mg of the desired oil type added and mixed with spatula. The mixture kept in the oil bath and mixed for 2 min every 20 min for total time of 4 hours. The vial let to cool at room temp for 1 hour

Determination of the oil content in the films: The films washed with 5 ml methanol for twice to remove the oil trace on the surface, then the films weighed and oil content calculated.

DSC: Differential scanning calorimetry (DSC) was performed to investigate the changes in the polymer thermal properties with the addition of different oils. The rate of thermal scan was fixed to 10° C. per minute, starting from 20° C. up to 125° C.

Oil extraction: The oil was extracted from the blends by placing the blends into 20 ml glass vial with 10 ml ethanol, the vials were placed on vortex for 2 days. Then the blends took out of the solvent vial, dried, and weighed. The solvent vial was placed in the hood for 2 days to evaporate all the ethanol and then the vials weighed to determine the amount of extracted oil

### Results:

The blends were prepared in the heat blending method by melting the PCL and then adding the oil while mixing for long time (3-5 hours). Different blends were prepared with different PCL molecular weight with different oil type. The amount of oil in the blends were affected by the MW of the PCL as seen in Table 13, the lowest oil loading was found in the PCL with MW=40 kDa and MW=3 kDa, the blends made with PCL with MW=80 kDa showed the highest oil loading for the different oil type, followed by the blends made with PCL MW=14 kDa. The MCT oil showed the highest loading upon the other oil in all the blends, followed by the castor oil, and the lowest oil loading was seen in the blends prepared with coconut as seen in Table 13. The blends made with PCL MW=80 kDa showed good mechanical properties.

The prepared blends with PCL MW=80 kDa were placed in ethanol to extract all the oils. The blends were weighed before and after the extraction experiment and the percentage of the oil in the sample were calculated based on the amount of oil extracted to compare it to the percentage before the experiment. The oils were completely extracted from the blends, as seen in Table 14. In addition, no observed change in the polymer material was found after the experiment. Spectral analysis, FTIR and NMR did not show new peaks but represented both polymer and the corresponding oil.

TABLE 23
oil ratio in the prepared blends with
PCL in different molecular weight

| | | Oil ratio in |
|---|---|---|
| Polymer | Oil type | the blend [w/w] |
| PCL 80 kDa | Castor | 30% |
| PCL 80 kDa | MCT | 39% |
| PCL 80 kDa | Coconut | 25% |
| PCL 40 kDa | Castor | 30% |
| PCL 40 kDa | MCT | 31% |
| PCL 40 kDa | Coconut | 12% |
| PCL 14 kDa | Castor | 27% |
| PCL 14 kDa | MCT | 34% |
| PCL 14 kDa | Coconut | 20% |
| PCL 3 kDa | Castor | 29% |
| PCL 3 kDa | MCT | 30% |
| PCL 3 kDa | Coconut | 12% |

TABLE 14
The blends weight and oil ratio before
and after the extraction using ethanol

| | Blend | Blend | | |
|---|---|---|---|---|
| | weight | weight | Total oil | Percentage oil |
| | before | after | content | extracted/ |
| Sample | [mg] | [mg] | before | ethanol wash |
| PCL-Blank | 26.62 | 26.49 | 0% | 0% |
| | 23.42 | 23.36 | 0% | 0% |
| PCL-Castor | 38.27 | 26.85 | 30% | 30% |
| | 31.58 | 22.11 | 32% | 30% |
| PCL-MCT | 41.68 | 25.72 | 39% | 38% |
| | 41.65 | 25.60 | 39% | 39% |

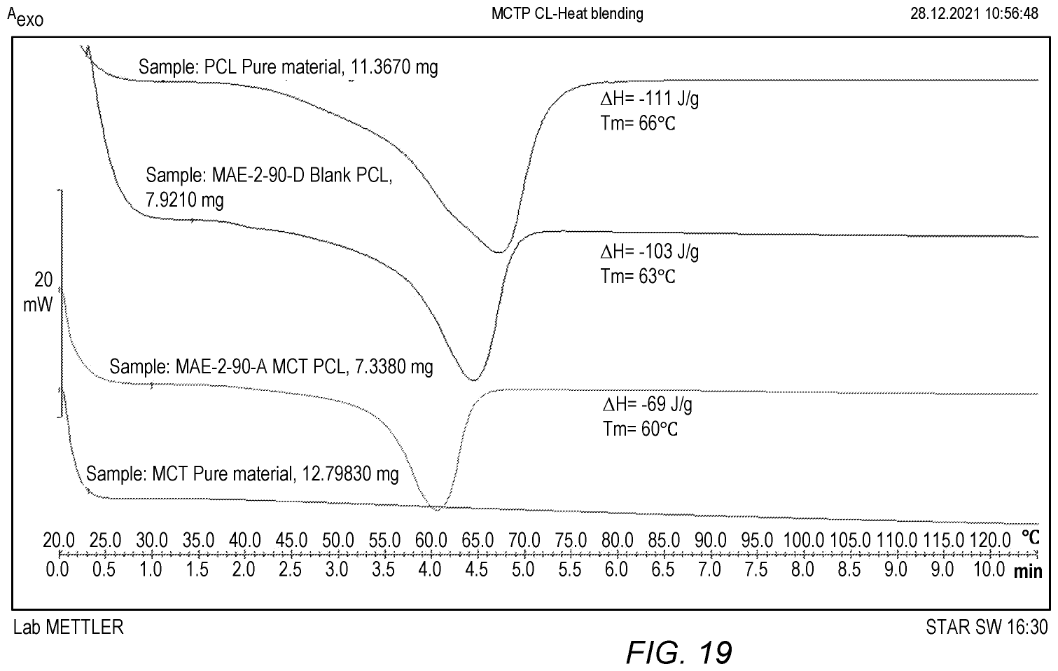

The DSC was performed on the blend made with PCL 80 kDa and MCT and compared to the Pure PCL 80 kDa and the blank PCL blend without oil. The DSC results shown in FIG. 19 demonstrated a decrease in the enthalpy in the PCL-MCT blend, in addition to decrease of the melting point, as seen the figure below.

Example 6: The blends of previous examples may contain a dispersed particulate solid such as silica, TiO2, ZnO, iron oxide, copper oxide, carbon nanotubes and graphene. In a typical experiment, to the solution of the lipid and polymer prepared in example 1, 10% w/w of nano-silica was added and mixed well to form a uniform dispersion which upon solvent evaporation a composite containing silica was obtained. Alternatively, the silica powder was added to the melt of the PCL-lipid composition and mixed well until it cools and solidifies. These compositions possess a higher mechanical strength than the material without the particles.

1. A homogeneous solid blend of at least one oil having a melting temperature below 50° C., and at least one polymer, the blend having at least one mechanical and/or physical property substantially identical to the mechanical and/or physical properties of the at least one polymer alone, wherein the solid blend is configured as an implantable fiducial marker.

2. (canceled)

3. (canceled)

4. The blend according to claim 1, wherein the blend is a homogeneous solid blend of at least one oil and at least one polymer, wherein the blend having a melting temperature that is substantially the same as the melting temperature of the at least one polymer when measured alone.

5. The blend according to claim 1, wherein the at least one polymer is a biodegradable polymer.

6. (canceled)

7. The blend according to claim 1, wherein the at least one polymer is an aliphatic polyester optionally selected from polyglycolide (PGA), polylactide (PLA), polycaprolactone (PCL), and their copolymers.

8. (canceled)

9. The blend according to claim 7, wherein the aliphatic polyester is poly(lactic-glycolic acid) (PLGA) or poly(lactic acid-caprolactone) copolymers (PLCL).

10. (canceled)

11. (canceled)

12. The blend according to claim 1, wherein the at least one oil is a naturally derived, a synthetic or a semisynthetic oil material.

13-16. (canceled)

17. The blend according to claim 12, wherein the at least one oil is an iodinated oil.

18-21. (canceled)

22. The blend according to claim 12, wherein the at least one oil is a fatty acid or a fatty alcohol or a fatty ester having a fatty saturated or unsaturated acid or alcohol carbon chain, or both, wherein the saturated or unsaturated acid or alcohol carbon chain having between 8 and 18 carbon atoms.

23-33. (canceled)

34. The blend according to claim 1, in a form selected from nanoparticles, microparticles, films, fibers, slabs, sheets, rods, plates, filaments, and shape memory compositions.

35. The blend according to any one of the preceding claim 1, comprising further at least one additive.

36-41. (canceled)

42. The blend according to claim 1, comprising at least one additive in a non-capsulated form.

43. The blend according to claim 1, wherein the blend comprises:
a hydrophilic drug in an encapsulated form;
a hydrophilic drug in an unencapsulated form;
a hydrophobic drug;
a hydrophobic drug in an encapsulated form;
a mixture of any of the above.

44-72. (canceled)

73. A clip or a biopsy marker formed of or comprising a homogeneous solid blend of at least one oil and at least one aliphatic polyester, wherein the blend having a melting temperature substantially identical to the melting temperature of the aliphatic polyester alone;
wherein the at least one oil is a naturally derived oil selected from triglycerides, fatty acids, fatty alcohols, fatty esters, vegetable oils and naturally occurring waxes; or a synthetic or a semisynthetic oil selected from triglycerides, fatty acids, fatty alcohols, fatty esters, paraffins, iodinated oils, modified oils, fully synthetic oils, hydrogenated vegetable oils and waxes; and wherein
the at least one polymer is a homopolymer or a copolymer of lactic acid, glycolic acid, caprolactone and di- and tri-methylene carbonate.

74. A process for manufacturing a solid blend according to claim 1, the process comprising treating a mixture of at least one oil and at least one polymer, under shear forces, optionally in the presence of a solvent, at a temperature between room temperature (25-30° C.) and a melting temperature of the at least one polymer.

75. The process according to claim 74, wherein the melting temperature of the at least one polymer is between 5° and 200° C.

76. (canceled)

77. (canceled)

78. The process according to claim 74, the process comprising treating a mixture of at least one oil and at least one polymer, in presence of a solvent, at a temperature between room temperature and the melting temperature of the at least one polymer, wherein the treating comprises application of shear forces to the mixture until complete or substantially complete evaporation of the solvent and formation of a homogenous blend, and cooling the blend to obtain the solid blend.

79-81. (canceled)

82. A solid homogeneous blend of at least one aliphatic polyester and at least one oil, the blend formed by applying a shear force to a mixture of the at least one aliphatic polyester and the at least one oil until a single-phase blend material is obtained, wherein the blend exhibits a melting temperature that is substantially same as the melting temperature of the at least one aliphatic polyester.

83. The blend according to claim 1, being a bioplastic configured for use in a method of manufacturing a biodegradable object.

84. The blend according to claim 83, wherein the object is selected from disposable syringes and medical supplies, boards, coatings, rods, 3D printing inks, containers, trays, sheets, foams and packaging materials.
